(19) United States
(12) Patent Application Publication
(10) Pub. No.: US 2026/0083561 A1
(43) Pub. Date: Mar. 26, 2026

(54) HEART VALVE REPAIR DEVICES AND DELIVERY DEVICES THEREFOR

(71) Applicant: Edwards Lifesciences Corporation, Irvine, CA (US)

(72) Inventors: Natan Anatoly Bukhdruker, Tirat Carmel (IL); Andrew Charles May, Irvine, CA (US); Jacob Timothy Morrison, Ladera Ranch, CA (US)

(21) Appl. No.: 19/408,262

(22) Filed: Dec. 3, 2025

Related U.S. Application Data
(63) Continuation of application No. PCT/US2024/032440, filed on Jun. 4, 2024.
(60) Provisional application No. 63/506,814, filed on Jun. 7, 2023.

(51) Int. Cl. A61F 2/24

(52) U.S. Cl. CPC A61F 2/2463; A61F 2/2466; A61F 2220/0008

(57) ABSTRACT

A valve repair device and valve repair systems including a delivery device and a valve repair device for repairing a native valve of a subject. The valve repair device and/or the delivery device having one or more force measurement devices for detecting a force applied to the native valve of a subject due to the connection between the valve repair device and the native valve. The valve repair device and the delivery system are used in a method of determining if a tension force provided to a native valve of a subject due to an attachment between a valve repair device and the native valve is at a desired level.

### RELATED APPLICATIONS

The present application is a continuation of Patent Cooperation Treaty Application No. PCT/US2024/032440, filed on Jun. 4, 2024, titled “Heart Valve Repair Devices and Delivery Devices Therefor”, which claims priority from U.S. Provisional patent application Ser. No. 63/506,814, filed on Jun. 7, 2023, titled “Heart Valve Repair Devices and Delivery Devices Therefor”, which are both incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves may be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In some implementations, there is provided a device or implant (e.g., implantable device, prosthetic device, etc.) that is configured to be positioned within a native heart valve to allow the native heart valve to form a more effective seal. The native heart valve can be a heart valve of a subject, such as of a living animal or a simulation.

In some implementations, an implantable device or implant includes one or more anchors. In some implementations, ach anchor includes a plurality of paddles that are each moveable between an open position and a closed position.

In some implementations, an example valve repair device includes at least one paddle, at least one gripping member and a force measurement device. In some implementations, the at least one paddle is movable between an open position and a closed position such that the at least one paddle is configured to attach the valve repair device to the native valve of a subject (e.g., of a living subject or of a simulation).

In some implementations, the at least one gripping member is configured to capture the native valve, and the force measurement device is attached to the at least one gripping member.

In some implementations, the force measurement device is configured to measure a force provided to the at least one gripping member when the valve repair device is attached to the native valve.

In some implementations, the at least one gripping member has a clasp that includes a fixed arm attached to the at least one paddle, a movable arm, and a hinge portion that hingeably connects the fixed arm to the movable arm.

In some implementations, the force measurement device is attached to the movable arm of the clasp.

In some implementations, the force measurement device is attached to the hinge portion of the clasp.

In some implementations, the force measurement device includes a strain gauge.

In some implementations, the force measurement device has a wire attached to the strain gauge, and the wire has a weakened portion that is configured to allow a user to break the wire after attaching the valve repair device to the native valve.

In some implementations, the at least one gripping member includes a first gripping member and a second gripping member, where the force measurement device is attached to the first gripping member and a second force measurement device is attached to the second gripping member.

In some implementations, the valve repair device includes an actuation element, where another force measurement device is attached to the actuation element.

In some implementations, the valve repair device includes a coaptation element.

In some implementations, the valve repair device includes a cap, where the at least one paddle is attached to the cap such that movement of the cap causes the at least one paddle to move between the open and closed positions.

In some implementations, the valve repair device includes a shaft and a coupler, where the coupler is attached to and movable along the shaft. The at least one paddle is attached to the coupler such that movement of the coupler along the shaft causes the at least one paddle to move between the open and closed positions.

In some implementations, the force measurement device is integrally formed with one or more components of the valve repair device.

In some implementations, the force measurement forms a portion of the at least one gripping member.

In some implementations, the force measurement device forms at least a portion of a hinge portion of the at least one gripping member.

In some implementations, the force measurement forms a portion of the at least one paddle.

In some implementations, the force measurement device forms at least a portion of a hinge portion of the at least one paddle.

In some implementations, an example valve repair system includes a delivery device and a valve repair device. In some implementations, the delivery device includes an actuation element and a force measurement device, where the force measurement device is attached to the actuation element.

In some implementations, the valve repair device includes a cap and at least one paddle that is attached to the cap.

In some implementations, the at least one paddle is movable between open and closed positions, and the at least one paddle is configured to attach the valve repair device to the native valve of a subject (e.g., of a living subject or of a simulation).

In some implementations, the actuation element is configured to engage the cap to move the cap and cause the at least one paddle to move between the open and closed positions.

In some implementations, the force measurement device is configured to detect a force applied to the actuation element when the valve repair device is attached to the native valve and the at least one paddle is in the closed position.

In some implementations, the force measurement device includes a strain gauge and a wire attached to the strain gauge.

In some implementations, the valve repair device includes at least one gripping member configured to capture the native valve of the subject.

In some implementations, the valve repair device includes a second force measurement device that is attached to the at least one gripping member, where the second force measurement device is configured to measure a force provided to the at least one gripping member when the valve repair device is attached to the native valve.

In some implementations, the at least one gripping member has a clasp that includes a fixed arm attached to the at least one paddle, a movable arm, and a hinge portion that hingeably connects the fixed arm to the movable arm.

In some implementations, the second force measurement device is attached to the movable arm of the clasp.

In some implementations, the second force measurement device is attached to the hinge portion of the clasp.

In some implementations, the second force measurement device includes a strain gauge.

In some implementations, the second force measurement device has a wire attached to the strain gauge, and the wire has a weakened portion that is configured to allow a user to break the wire after attaching the valve repair device to the native valve.

In some implementations, the at least one gripping member includes a first gripping member and a second gripping member, where the second force measurement device is attached to the first gripping member and a third force measurement device is attached to the second gripping member.

In some implementations, an example valve repair system includes a delivery device and a valve repair device. In some implementations, the delivery device includes a paddle control mechanism and a force measurement device, where the force measurement device is attached to the paddle control mechanism.

In some implementations, the valve repair device includes a shaft, a coupler, and at least one paddle that is attached to the coupler.

In some implementations, the at least one paddle is movable between open and closed positions, and the at least one paddle is configured to attach the valve repair device to the native valve of a subject (e.g., of a living subject or of a simulation).

In some implementations, the paddle control mechanism of the delivery device is configured to engage the coupler to move the coupler along the shaft and cause the at least one paddle to move between the open and closed positions.

In some implementations, the force measurement device is configured to detect a force applied to the paddle control mechanism when the valve repair device is attached to the native valve and the at least one paddle is in the closed position.

In some implementations, the force measurement device includes a strain gauge and a wire attached to the strain gauge.

In some implementations, the valve repair device includes at least one gripping member configured to capture the native valve of the subject.

In some implementations, the valve repair device includes a second force measurement device that is attached to the at least one gripping member, where the second force measurement device is configured to measure a force provided to the at least one gripping member when the valve repair device is attached to the native valve.

In some implementations, the at least one gripping member has a clasp that includes a fixed arm attached to the at least one paddle, a movable arm, and a hinge portion that hingeably connects the fixed arm to the movable arm.

In some implementations, the second force measurement device is attached to the movable arm of the clasp.

In some implementations, the second force measurement device is attached to the hinge portion of the clasp.

In some implementations, the second force measurement device includes a strain gauge.

In some implementations, the second force measurement device has a wire attached to the strain gauge, and the wire has a weakened portion that is configured to allow a user to break the wire after attaching the valve repair device to the native valve.

In some implementations, the at least one gripping member includes a first gripping member and a second gripping member, where the second force measurement device is attached to the first gripping member and a third force measurement device is attached to the second gripping member.

In some implementations, an example method for determining if a tension force provided to a native valve of a subject (e.g., of a living animal or a simulation) due to an attachment between a valve repair device and the native valve is at a desired level includes delivering the valve repair device to the native valve of the subject with a delivery device, where at least one of the valve repair device and the delivery device include a force measurement device.

In some implementations, the example method further includes attaching the valve repair device to the native valve of the subject and detecting a force applied to at least one of the valve repair device and the delivery device via the force measurement device.

In some implementations, the example method further includes determining if a tension force applied to the native valve is at a desired level based on the detected force by the force measurement device.

In some implementations, the example method includes detaching the valve repair device from the native valve if the tension force is not at a desired level and reattaching the valve repair device to the native valve.

In some implementations, the example method can further includes redetecting the force applied to at least one of the valve repair device and the delivery device via the force measurement device and determining if the tension force applied to the leaflets is at a desired level based on the redetected force by the force measurement device.

In some implementations, the valve repair device includes at least one paddle and at least one gripping member, where the force measurement device is attached to the at least one gripping member.

In some implementations, the at least one gripping member has a clasp that includes a fixed arm attached to the at least one paddle, a movable arm, and a hinge portion that hingeably connects the fixed arm to the movable arm.

In some implementations, the second force measurement device is attached to the movable arm of the clasp.

In some implementations, the second force measurement device is attached to the hinge portion of the clasp.

In some implementations, the second force measurement device includes a strain gauge.

In some implementations, the detected force is detected via an ohmmeter.

In some implementations, the delivery device comprises an actuation element, and the force measurement device is attached to the actuation element.

In some implementations, the valve repair device has a cap and at least one paddle attached to the cap, where the at least one paddle is configured to attach the valve repair device to the native valve.

In some implementations, the actuation element can be configured to engage the cap to move the cap and cause the at least one paddle to move between the open and closed positions.

In some implementations, the delivery device has a paddle control mechanism, and the force measurement device is attached to the paddle control mechanism.

In some implementations, the valve repair device has a shaft, a coupler that is attached to and movable along the shaft, and at least one paddle attached to the coupler.

In some implementations, the at least one paddle can be movable between open and closed positions, and the at least one paddle can be configured to attach the valve repair device to the native valve.

In some implementations, the paddle control mechanism can be configured to engage the coupler to move the coupler along the shaft and cause the at least one paddle to move between the open and closed positions.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with subjects, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a perspective view of an example implantable device or implant in a closed position;

FIG. 24 illustrates an example valve repair device with paddles in an open position;

FIG. 25A illustrates another example valve repair device with paddles in a closed position;

FIG. 25B illustrates a top view of an example valve repair device;

FIG. 26 illustrates a perspective view of an example implantable device having paddles of adjustable widths;

FIG. 27 is a cross-section of the implantable device of FIG. 26 in which the implantable device is bisected;

FIG. 28 is another cross-section of the implantable device of FIG. 26 in which the implantable device is bisected along a plane perpendicular to the plane illustrated in FIG. 28;

FIG. 29 is a schematic illustration of an example implant catheter assembly coupled to an implantable device in which an actuation element is coupled to a paddle actuation control and to a driver head of the implantable device;

FIG. 30 is an illustration of the assembly of FIG. 29 with the implantable device rotated 90 degrees to show the paddle width adjustment element coupled to an inner end of a connector of the implantable device and coupled to a paddle width control;

FIG. 31 shows a partial view of an example of an implantable device or implant that is similar to the device illustrated by FIGS. 8-14, but where an example force measurement device is disposed on or in a delivery system for delivering the implantable device or implant;

FIG. 32 shows a partial view of an example of an implantable device or implant that is similar to the device illustrated by FIGS. 8-14, but where example force measurement devices are disposed on the implantable device or implant;

FIG. 33 shows an example of an implantable device or implant that is similar to the device illustrated by FIG. 22, but where example force measurement devices are disposed on at least one of the implantable device or implant and a delivery system for delivering the implantable device or implant;

FIG. 34 shows an example of an implantable device or implant that is similar to the device illustrated by FIG. 23, but where example force measurement devices are disposed on at least one of the implantable device or implant and a delivery system for delivering the implantable device or implant;

FIG. 35 shows an example of an implantable device or implant that is similar to the device illustrated by FIG. 24, but where example force measurement devices are disposed on at least one of the implantable device or implant and a delivery system for delivering the implantable device or implant;

FIG. 36 shows an example of an implantable device or implant that is similar to the device illustrated by FIG. 25A, but where an example force measurement device is disposed on the implantable device or implant;

FIGS. 37-39 shows an example of an implantable device or implant that is similar to the device illustrated by FIGS. 26-28, but where example force measurement devices are disposed on at least one of the implantable device or implant and an implant catheter assembly for delivering the implantable device or implant;

FIG. 40 shows a partial view of an example of an implantable device or implant that is similar to the device illustrated by FIGS. 8-14, but where example force measurement devices are integrally formed with one or more components of the implantable device or implant;

FIG. 41 shows an example of an implantable device or implant that is similar to the device illustrated by FIG. 22, but where example force measurement devices are integrally formed with one or more components of the implantable device or implant;

FIG. 42 shows an example of an implantable device or implant that is similar to the device illustrated by FIG. 23, but where example force measurement devices are integrally formed with one or more components of the implantable device or implant;

FIG. 43 shows an example of an implantable device or implant that is similar to the device illustrated by FIG. 24, but where example force measurement devices are integrally formed with one or more components of the implantable device or implant; and

FIG. 44 shows an example of an implantable device or implant that is similar to the device illustrated by FIGS. 26-28, but where example force measurement devices are integrally formed with one or more components of the implantable device or implant.

### DETAILED DESCRIPTION

The following description refers to the accompanying drawings, which illustrate some implementations of the present disclosure. Some implementations having different structures and operation do not depart from the scope of the present disclosure.

Example implementations of the present disclosure are directed to systems, devices, methods, etc. for repairing a defective heart valve. For example, some implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques and methods herein can be performed on a living animal or on a simulation, such as on a cadaver, cadaver heart, simulator (e.g., with the body parts, heart, tissue, etc. being simulated), imaginary person (e.g., demonstrating in the air on an imaginary heart), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

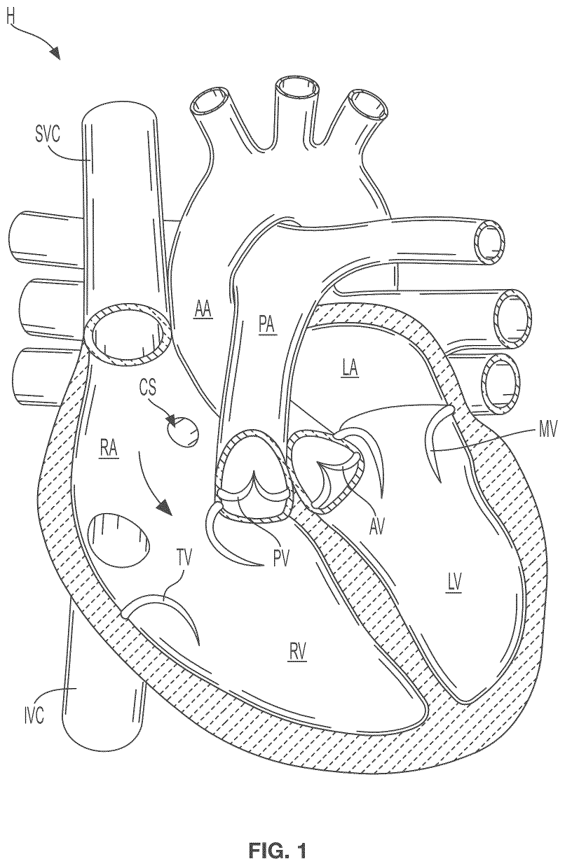

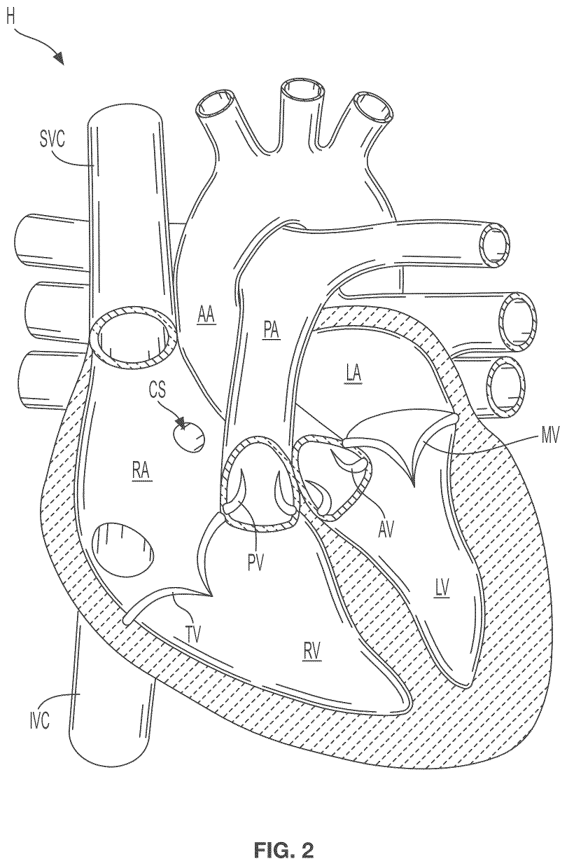

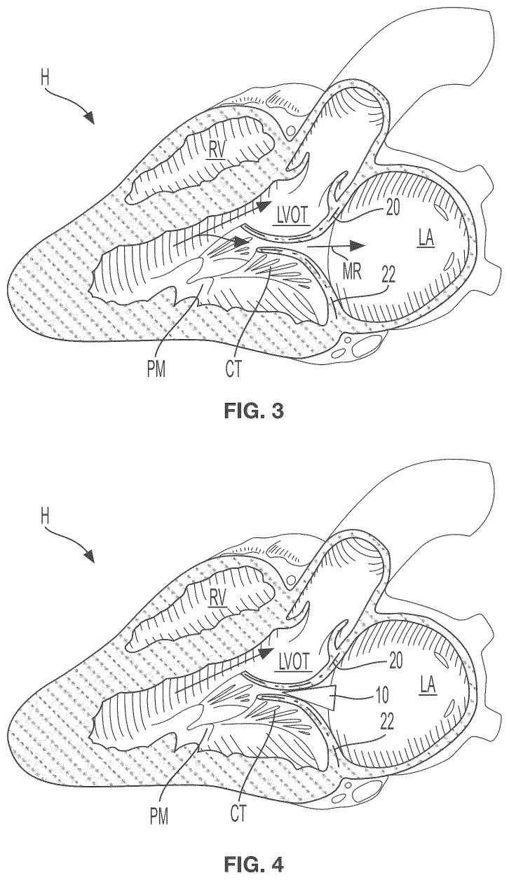

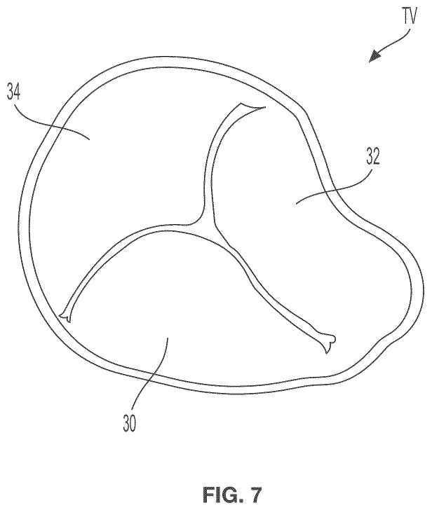

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

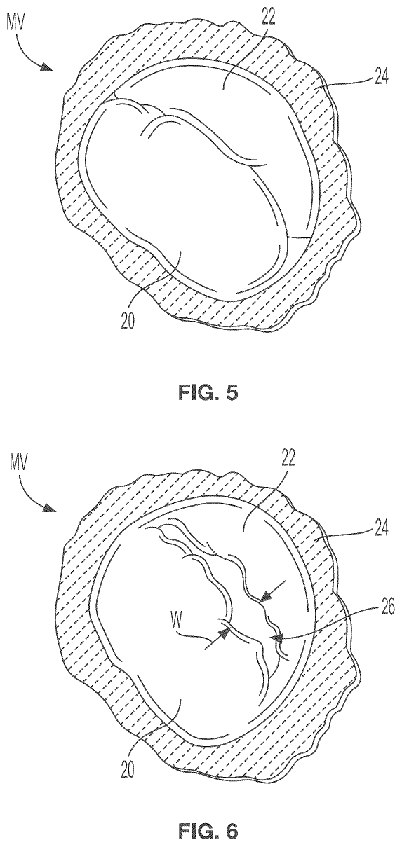

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of subjects undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm or even 17.5 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of an implantable device, valve repair device, or implant 10 is shown implanted between the leaflets 20, 22 such that regurgitation does not occur during systole (compare FIG. 3 with FIG. 4). In some implementations, the coaptation element (e.g., spacer, coaption element, gap filler, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another). In some implementations, the coaptation element or spacer is optional in any of the valve repair devices disclosed herein.

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the subject's native valve. Replacement typically involves replacing the subject's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implantable device or implant can optionally have a coaptation element (e.g., spacer, coaption element, gap filler, etc.) and at least one anchor (e.g., one, two, three, or more). In some implementations, an implantable device or implant can have any combination or sub-combination of the features disclosed herein without a coaptation element. When included, the coaptation element (e.g., spacer, coaption element, gap filler, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still-compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for some implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

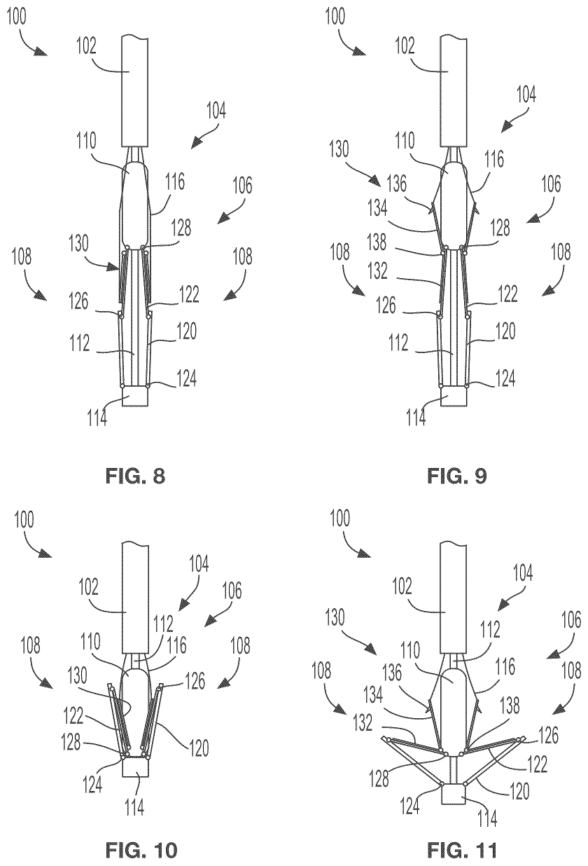

Referring now to FIGS. 8-15, a schematically illustrated implantable device or implant 100 (e.g., an implantable prosthetic device, a prosthetic spacer device, a valve repair device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for an implantable device or implant discussed in the present application or the applications cited above, and the device 100 can be positioned to engage valve tissue (e.g., leaflets 20, 22, 30, 32, 34) as part of any suitable valve repair system (e.g., any valve repair systems/devices disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 that is adapted to be implanted between leaflets of a native valve (e.g., a native mitral valve, native tricuspid valve, etc.) and is slidably attached to an actuation element 112 (e.g., actuation wire, actuation shaft, actuation tube, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, actuation shaft, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., coupler, clamp, removable fastener, clip, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members. The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other optional friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.). The clasps 130 can be used to grasp and/or further secure the native leaflets by engaging the leaflets with optional barbs or other optional friction-enhancing elements 136 and pinching the leaflets between the moveable and fixed arms 134, 132. The barbs or other friction-enhancing elements 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable portions 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, actuation shaft, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., wire, shaft, etc.) and cap (or other attachment portion) are used to control one paddle, and the other independent actuation element and cap (or other attachment portion) are used to control the other paddle.

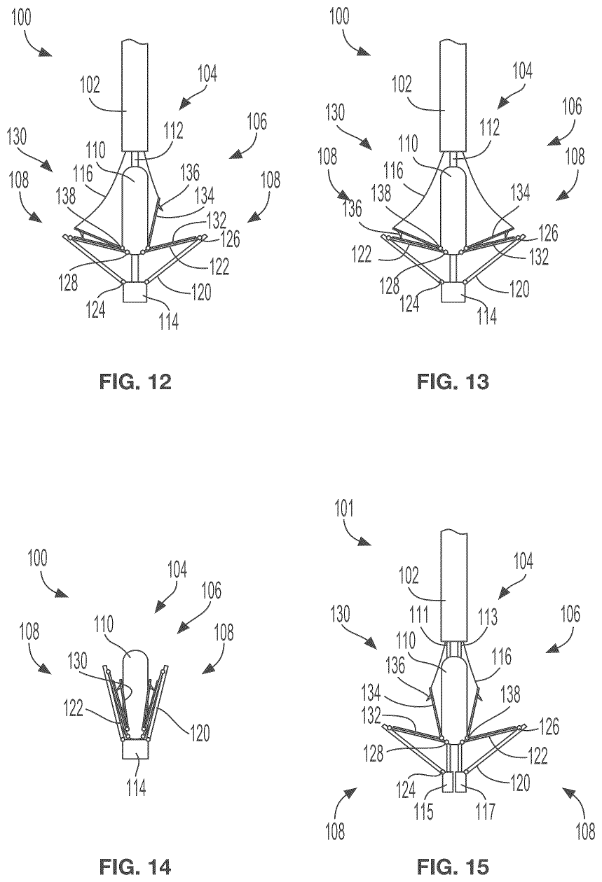

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 101 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

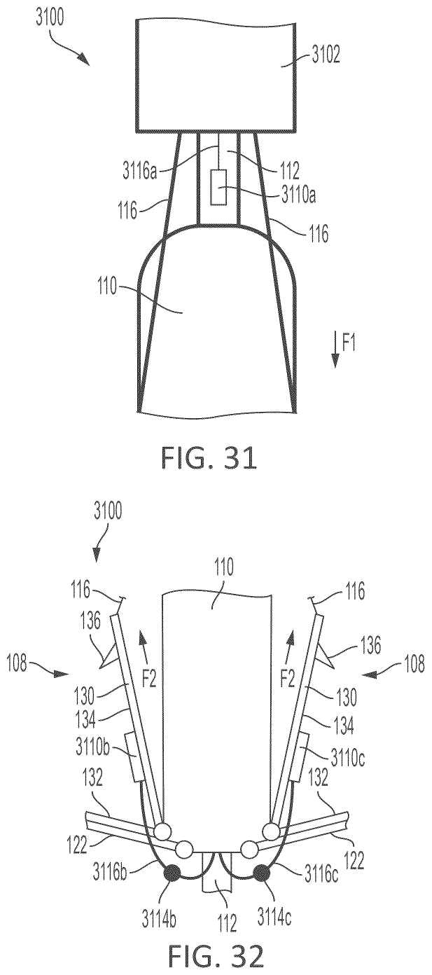

FIGS. 31 and 32 illustrate an example of a device 3100 and/or delivery system 3102 that are configured to detect a tension force provided to the leaflets due to the connection between the device 3100 and the leaflets. The device 3100 and delivery system 3102 illustrated by FIGS. 31 and 32 can be the same as or similar to the device 100 and delivery system 102 illustrated by FIGS. 8-14. In some implementations, the device 3100 and/or delivery system 3102 of FIGS. 31 and 32 include one or more force measurement devices 3110a-3110c for detecting a tension force provided to the leaflets. That is, the connection of the device 3100 with the leaflets causes a tension force to be applied to the leaflets, and the force measurement devices 3110a-3110c can be configured to measure the tension force provided to the leaflets such that a user can determine if the tension force is higher or lower than an optimal tension force range. For example, if the tension force is below a predetermined amount, there may be a poor attachment between the device 3100 and the leaflets. If the tension force is above a predetermined amount, too much of the leaflets may be grasped by the device 3100.

The force measurement devices 3110a-3110c can take a variety of different forms. For example, the force measurement devices 3110a-3110c can be one or more of a strain gauge, an optical sensor, a proximity sensor, and/or a hall sensor, etc.

The force measurement device(s) can be disposed at any location on the device 3100 and/or delivery system 3102 that allows for the force measurement device(s) to measure a tension force provided to the leaflets and/or to measure a tension force, another force, position, etc. that correlates to tensile force on the leaflets. For example, referring to FIG. 31, a force measurement device 3110a can be disposed on an actuation element 112 of the delivery system 3102.

In some implementations, actuation of the actuation element 112 opens and closes the one or more anchors 108 of the device 3100 to grasp the native valve leaflets during implantation. For example, as described herein with reference to FIGS. 8-14, extension of the actuation element 112 causes a cap (e.g., cap 114) to move away from the coaptation element 110, which moves one or more anchors 108 to the open position, and retraction of the actuation element 112 causes the cap to move toward the coaptation element 110, which moves the one or more anchors 108 to the closed position.

When the valve repair device 3100 is attached to the leaflets and in the closed position, this attachment between the valve repair device 3100 and the leaflets can cause a tension force on the leaflets and a corresponding force on the one or more anchors 108. In some circumstances, this force provided to the anchors 108 can potentially cause the anchors to partially open, which may cause a force F1 on the actuation element 112 because the anchors 108 open and close due to movement of the actuation element 112. In some implementations, the force measurement device 3110a is configured to measure the force F1 on the actuation element 112, and the measurement of the force F1 allows a user to determine if the corresponding tension force provided to the leaflets is at a desired or undesired level.

In the illustrated example, the force measurement device 3110a includes a strain gauge having one or more wires 3116a that extend to a device (not shown) for measuring electrical resistance (e.g., an ohmmeter). In some implementations, after the valve repair device 3100 is attached to the native valve and the tension force provided to the leaflets is determined to be at a desired level, the actuation element 112 is detached from the valve repair device 3100 and removed from the subject with the delivery system 3102.

In some implementations, the force measurement device 3110a can be attached to a portion of the actuation element 112 that is integral to the valve repair device 3100. In these examples, the wire 3116a of the strain gauge can include a weakened portion (e.g., similar to the weakened portions 3114b, 3114c described with reference to FIG. 32) that allows for the wire 3116a to be broken such that the delivery system 3102 can be removed from the subject while the strain gauge remains on the valve repair device 3100 that is implanted in the subject.

Referring to FIG. 32, in some instances, a force measurement device 3110b can be placed on one gripping member of the valve repair device 3100 and another force measurement device 3110c can be placed on another gripping member of the valve repair device 3100. While the illustrated example shows a valve repair device 3100 having two gripping members, it should be understood that the valve repair device 3100 can have any suitable number of gripping members, clasps, etc., and it should be understood that a force measurement device can be disposed on one or more of the gripping members/clasps/etc. In the illustrated example, the force measurement devices 3110b, 3110c are disposed on movable arms 134 of clasps 130 of the gripping members, but it should be understood that the force measurement devices 3110b, 3110c can be positioned at any location on the gripping members that allows for the force measurement devices 3110b, 3110c to measure the tension force provided to the leaflets.

When the valve repair device 3100 is attached to the leaflets and in the closed position, this attachment between the valve repair device 3100 and the leaflets can cause a tension force on the leaflets and a corresponding force F2 to the gripping members. In some implementations, the force measurement devices 3110b, 3110c are configured to measure the force F2 on the corresponding gripping members, and the measurement of the force F2 allows a user to determine if the corresponding tension force provided to one or more of the leaflets is at a desired or undesired level.

In some implementations, each of the force measurement devices 3110b, 3110c can include a strain gauge that has one or more corresponding wires 3116b, 3116c that extend to a device for measuring electrical resistance or another electrical property that can be used to measure force and/or strain. In some implementations, the wires 3116b, 3116c can each have a separation feature, such as a connector or weakened portion 3114b, 3114c that allows for the wires to be broken and/or separated such that the strain gauges can remain on the valve repair device 3100, and such that at least a portion of each of the wires 3116b, 3116c can be removed with the delivery system 3102 from the subject. In some implementations, the weakened portions 3114b, 3114c can be, for example, a soldered joint, a thinner portion of the wire, a thin portion of a printed circuit trace, a temporary mechanical connection, such as a twisted wire connection, an electrical connector, such as a plug and/or socket, or any other suitable means of allowing for a user to separate and/or break the wires 3116b, 3116c after implantation of the valve repair device 3100. In some examples, a cutting device (not shown) can be used to cut the wires 3116b, 3116c to break the wires.

While the force measurement devices 3110a-3110c are described as including a strain gauge, it should be understood that the force measurement devices 3110a-3110c can take any other suitable form that is capable of determining a tension force applied to the leaflets by the valve repair device 3100. For example, the force measurement devices 3110a-3110c can include a load cell sensor, a strainpad, a piezoelectric sensor, any suitable positional sensor for detecting change in a position of the sensor due to forces created by the connection between the valve repair device and the native valve, or any other suitable type of sensor that is capable of allowing a user to determine a tension force provided to one or more leaflets of the native valve.

While the illustrated examples shown in FIGS. 31 and 32 show the valve repair device 3100 and the delivery system 3102 having three force measurement devices 3110a-3110c, it should be understood that the valve repair device 3100 and/or the delivery system 3102 can have any suitable number of force measurement devices. In some instances, only the valve repair device 3100 includes one or more force measurement devices. In some instances, only the delivery device 3102 includes one or more force measurement devices.

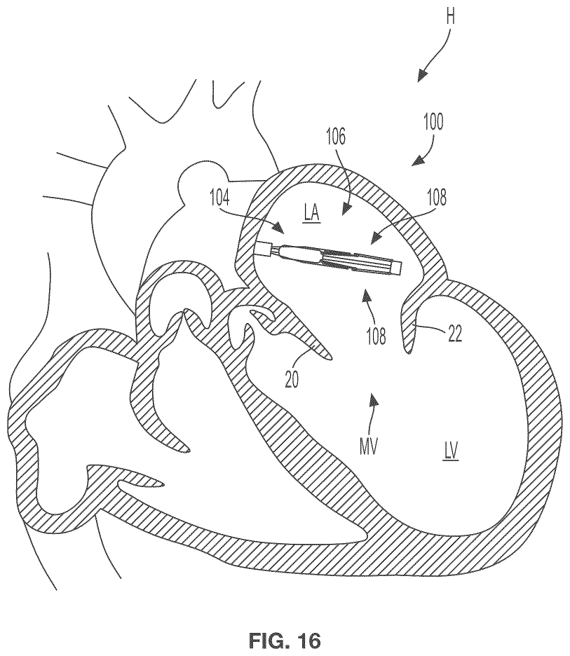

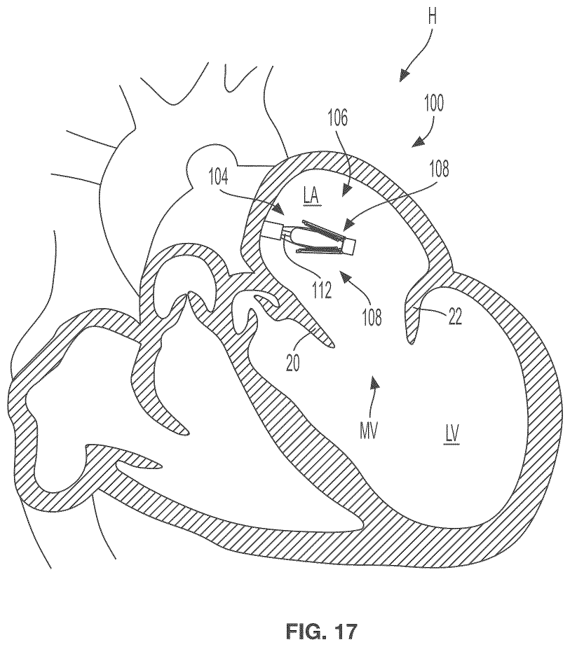

Referring now to FIGS. 16-21, the implantable device 100 of FIGS. 8-14 is shown being delivered and implanted within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

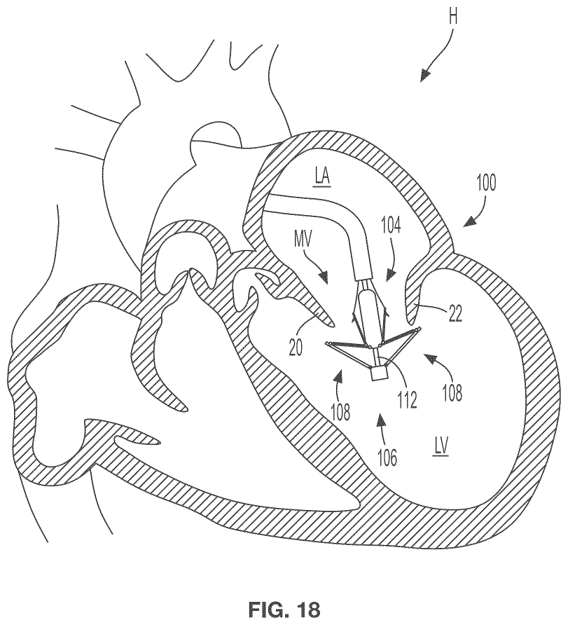

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

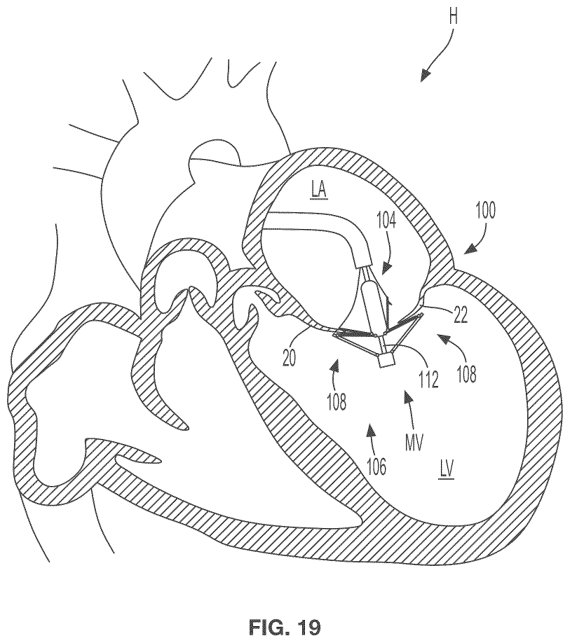

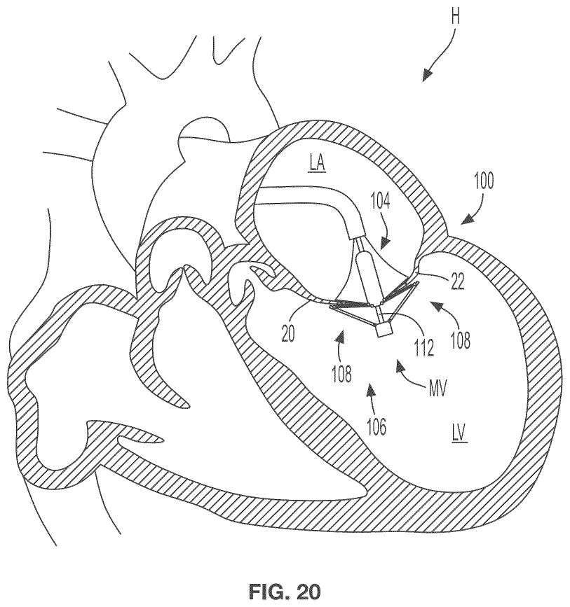

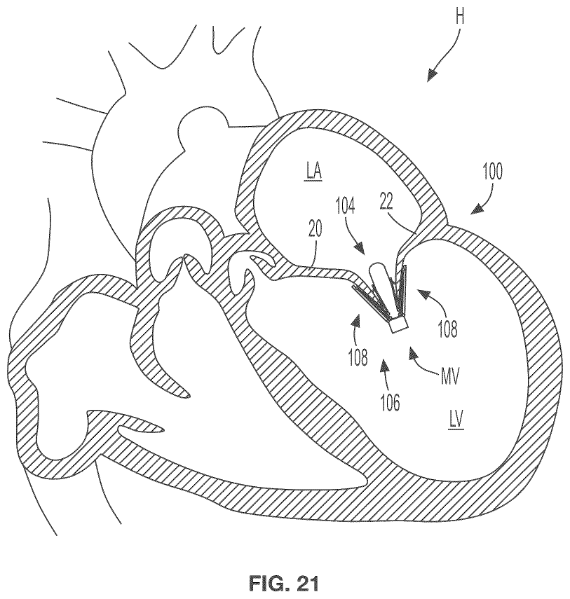

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

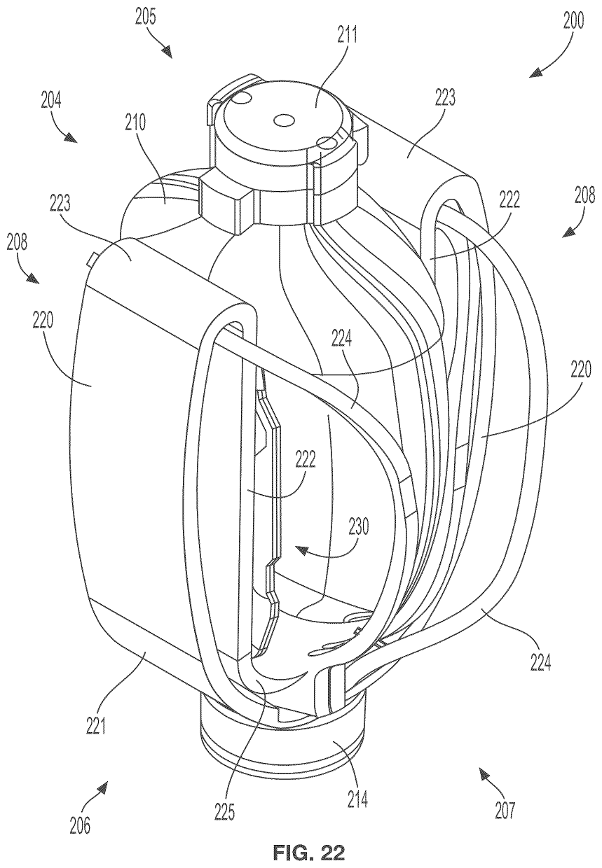

Any of the features disclosed by the present application can be used in a wide variety of different valve repair devices. FIGS. 22-24 illustrate examples of valve repair devices that can be modified to include any of the features disclosed by the present application. Any combination or sub-combination of the features disclosed by the present application can be combined with, substituted for, and/or added to any combination or sub-combination of the features of the valve repair devices illustrated by FIGS. 8-24.

Referring now to FIG. 22, an example of an implantable device or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

In some implementations, the implantable device or implant 200 includes a coaptation portion 204, a proximal or attachment portion 205, an anchor portion 206, and a distal portion 207. In some implementations, the coaptation portion 204 of the device optionally includes a coaptation element 210 (e.g., a spacer, coaption element, plug, membrane, sheet, gap filler, etc.) 210 for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 205 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the implantable device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single subject will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member (e.g., bar, strut, etc.) or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215). Any combination or sub-combination of the features disclosed by the present application can be combined with any combination or sub-combination of the features disclosed by Patent Cooperation Treaty International Application No. PCT/US2018/028189 (International Publication No. WO 2018/195215). Patent Cooperation Treaty International Application No. PCT/US2018/028189 (International Publication No. WO 2018/195215) is incorporated herein by reference in its entirety.

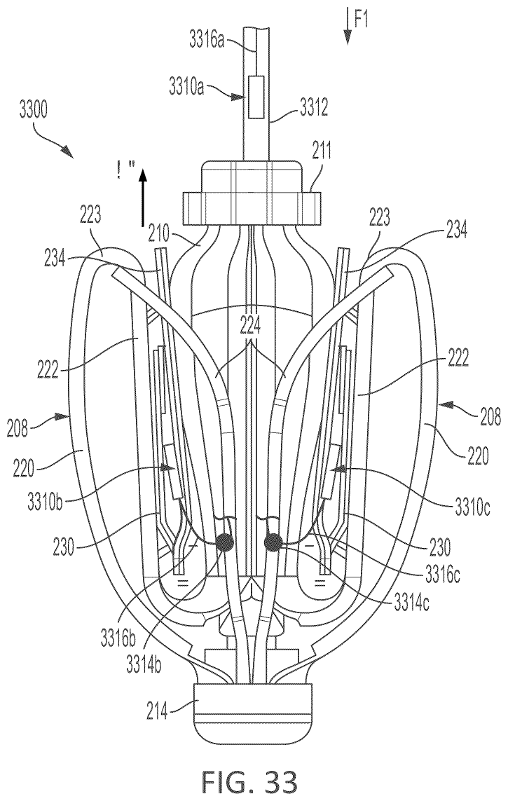

FIG. 33 illustrates an example of a device 3300 and/or an actuation element 3312 of a delivery system that are configured to detect a tension force provided to the leaflets due to the connection between the device 3300 and the leaflets. The device 3300 can be the same as or similar to the device 200 illustrated by FIG. 22. In some implementations, the device 3300 of FIG. 33 includes one or more force measurement devices 3310a-3310c for detecting a tension force provided to the leaflets. That is, the connection of the device 3300 with the leaflets causes a tension force on the leaflets, and the force measurement devices 3310a-3310c can be configured to measure the tension force provided to the leaflets such that a user can determine if the tension force is higher or lower than optimal force range. For example, if the tension force is below a predetermined amount, there may be a poor attachment between the device 3300 and the leaflets. If the tension force is above a predetermined amount, too much of the leaflets may be grasped by the device 3300.

The force measurement devices 3110a-3110c can take a variety of different forms. For example, the force measurement devices 3110a-3110c can be one or more of a strain gauge, an optical sensor, a proximity sensor, and/or a hall sensor, etc.

The force measurement device(s) can be disposed at any location on the device 3300 and/or delivery system that allows for the force measurement device(s) to measure a tension force provided to the leaflets and/or to measure a tension force, another force, position, etc. that correlates to tensile force on the leaflets. In the illustrated example, a force measurement device 3310a is disposed on an actuation element 3312 of the delivery system.

In some implementations, actuation of the actuation element 3312 opens and closes the one or more anchors 208 of the device 3300 to grasp the native valve leaflets during implantation. For example, extension of the actuation element 3312 can cause the cap 214 of the device 3300 to move away from the coaptation element 210, which moves one or more anchors 208 to the open position, and retraction of the actuation element 3312 can cause the cap 214 to move toward the coaptation element 210, which moves the one or more anchors 208 to the closed position.

When the valve repair device 3300 is attached to the leaflets and in the closed position, this attachment between the valve repair device 3300 and the leaflets can cause a tension force on the leaflets and a corresponding force on the one or more anchors 208. This force provided to the anchors 208 can potentially cause the anchors to partially open, which causes a force F1 on the actuation element 3312 because the anchors 208 open and close due to movement of the actuation element 3312. The force measurement device 3310a is configured to measure the force F1 on the actuation element 3312, and the measurement of the force F1 allows a user to determine if the corresponding tension force provided to the leaflets is at a desired or undesired level.

In the illustrated example, the force measurement device 3310a includes a strain gauge having one or more wires 3316a that extend to a device (not shown) for measuring electrical resistance (e.g., an ohmmeter). After the valve repair device 3300 is attached to the native valve and the tension force provided to the leaflets is determined to be at a desired level, the actuation element 3312 is detached from the valve repair device 3300 and removed from the subject with the delivery system. In other examples, the force measurement device 3310a may be attached to a portion of the actuation element 3312 that is integral to the valve repair device 3300. In these examples, the wire 3316a of the strain gauge can include a weakened portion (e.g., similar to the weakened portions 3314b, 3314c of the wires 3316b, 3316c of the force measurement devices 3310b, 3310c) that allows for the wire 3316a to be broken and/or separated such that the delivery system can be removed from the subject while the strain gauge remains on the valve repair device 3300 that is implanted in the subject.

In some examples, a force measurement device 3310b can be placed on one clasp 230 of the valve repair device 3300 and another force measurement device 3310c can be place on another clasp 230 of the valve repair device 3300. While the illustrated example shows a valve repair device 3300 having two clasps 230, it should be understood that the valve repair device 3300 can have any suitable number of clasps, and it should be understood that a force measurement device can be disposed on one or more of the clasps. In the illustrated example, the force measurement devices 3310b, 3310c are disposed on movable arms 234 of the clasps 230, but it should be understood that the force measurement devices 3310b, 3310c can be positioned at any location on the clasps 230 that allows for the force measurement devices 3310b, 3310c to measure the tension force provided to the leaflets.

When the valve repair device 3300 is attached to the leaflets and in the closed position, this attachment between the valve repair device 3300 and the leaflets can cause a tension force on the leaflets and a corresponding force F2 to the clasps 230. The force measurement devices 3310b, 3310c are configured to measure the force F2 on the corresponding gripping members, and the measurement of the force F2 allows a user to determine if the tension force provided to one or more of the leaflets is at a desired or undesired level.

In some implementations, each of force measurement devices 3310b, 3310c can include a strain gauge that has one or more corresponding wires 3316b, 3316c that extend to a device for measuring electrical resistance. In some implementations, the wires 3316b, 3316c can each have a weakened portion 3314b, 3314c that allows for the wires to be separated and/or broken such that the strain gauges can remain on the valve repair device 3300, and such that at least a portion of each of the wires 3316b, 3316c can be removed with the delivery system from the subject.

In some implementations, the weakened portions 3314b, 3314c can be, for example, a soldered joint, a thinner portion of the wire, a thin portion of a printed circuit trace, a temporary mechanical connection, such as a twisted wire connection, an electrical connector, such as a plug and/or socket, or any other suitable means of allowing for a user to break the wires 3316b, 3316c after implantation of the valve repair device 3300. In some examples, a cutting device (not shown) may be used to cut the wires 3316b, 3316c to break the wires.

While the force measurement devices 3310a-3310c are described as including a strain gauge, it should be understood that the force measurement devices 3310a-3310c can take any other suitable form that is capable of determining a tension force applied to the leaflets by the valve repair device 3300 and/or to measure a tension force, another force, position, etc. that correlates to tensile force on the leaflets. For example, the force measurement devices 3310a-3310c can include a load cell sensor, a strainpad, a piezoelectric sensor, any suitable positional sensor for detecting change in a position of the sensor due to forces created by the connection between the valve repair device and the native valve, or any other suitable type of sensor that is capable of allowing a user to determine and/or or correlate a tension force provided to one or more leaflets of the native valve.

While the illustrated example shown in FIG. 33 shows the valve repair device 3300 and the delivery system having three force measurement devices 3310a-3310c, it should be understood that the valve repair device 3300 and/or the delivery system can have any suitable number of force measurement devices. In some instances, only the valve repair device 3300 includes one or more force measurement devices. In other instances, only the delivery device includes one or more force measurement devices.

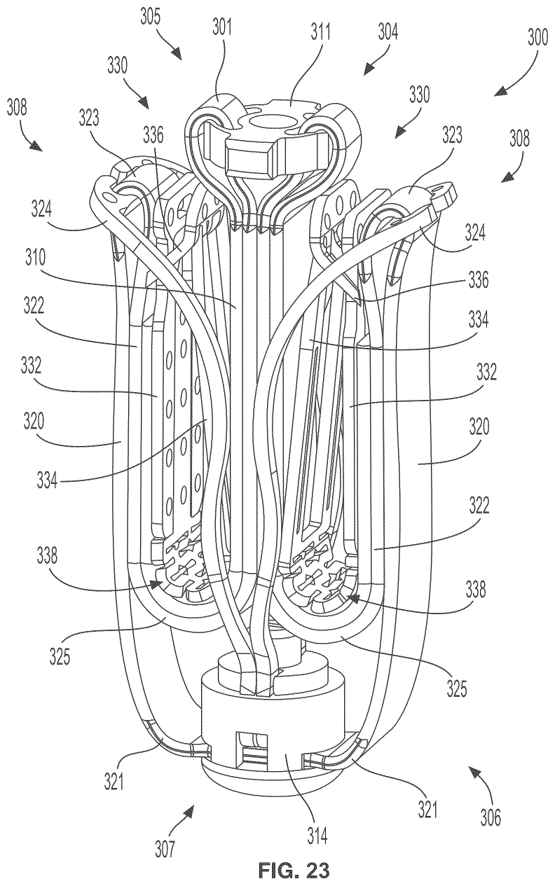

Referring now to FIG. 23, an example of an implantable device or implant 300 is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

In some implementations, the implantable device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve.

In some implementations, the anchor portion 306 includes a plurality of anchors 308. In some implementations, each anchor 308 can include one or more paddles, e.g., outer paddles 320, inner paddles 322, paddle extension members (e.g., leaf spring, shaped wire, etc.) or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

In some implementations, the anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In some implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338.

In some implementations, the fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

In some implementations, the fixed arms 332 are attached to the inner paddles 322 through holes or slots with sutures. In some implementations, the fixed arms 332 can be attached to the inner paddles 322 with any suitable means, such as screws or other fasteners, crimped sleeves, mechanical latches or snaps, welding, adhesive, or the like.

In some implementations, the fixed arms 332 remain substantially stationary relative to the inner paddles 322 when the moveable arms 334 are opened to open the clasps 330 and expose the optional barbs 336.

In some implementations, the clasps 330 are opened by applying tension to actuation lines attached to the moveable arms 334, thereby causing the moveable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301.

In some implementations, the continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. In the illustrated example of FIG. 23, the strip of material 301 begins and ends in the location of the inner paddles 322.

As with the implantable device or implant 200 described above, the size of the coaptation element 310 can be selected to minimize the number of implants that a single subject will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety.

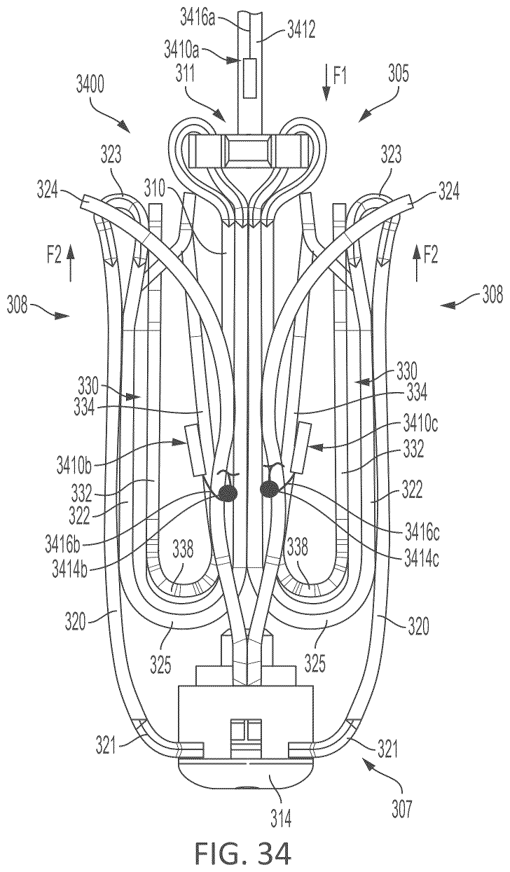

FIG. 34 illustrates an example of a device 3400 and/or an actuation element 3412 of a delivery system that are configured to detect a tension force provided to the leaflets due to the connection between the device 3400 and the leaflets. The device 3400 can be the same as or similar to the device 300 illustrated by FIG. 23. In some implementations, the device 3400 of FIG. 34 includes one or more force measurement devices 3410a-3410c for detecting a tension force provided to the leaflets and/or to measure a tension force, another force, position, etc. that correlates to tensile force on the leaflets.

In some implementations, the connection of the device 3400 with the leaflets causes a tension force to be applied to the leaflets, and the force measurement devices 3410a-3410c can be configured to measure the tension force provided to the leaflets and/or to measure a force that can be correlated to the tension force applied to the leaflets such that a user can determine if the tension force is higher or lower than an optimal tension force range. For example, if the tension force is below a predetermined amount, there may be a poor attachment between the device 3400 and the leaflets. If the tension force is above a predetermined amount, too much of the leaflets may be grasped by the device 3400.

The force measurement device(s) can be disposed at any location on the device 3400 and/or delivery system that allows for the force measurement device(s) to measure a tension force provided to the leaflets. In the illustrated example, a force measurement device 3410a is disposed on an actuation element 3412 of the delivery system. In some implementations, actuation of the actuation element 3412 opens and closes the one or more anchors 308 of the device 3400 to grasp the native valve leaflets during implantation. For example, extension of the actuation element 3412 can cause the cap 314 of the device 3400 to move away from the coaptation element 310, which moves one or more anchors 308 to the open position, and retraction of the actuation element 3412 can cause the cap 314 to move toward the coaptation element 310, which moves the one or more anchors 308 to the closed position.

When the valve repair device 3400 is attached to the leaflets and in the closed position, this attachment between the valve repair device 3400 and the leaflets can cause a tension force on the leaflets and a corresponding force on the one or more anchors 308. This force provided to the anchors 308 could potentially cause the anchors to partially open, which causes a force F1 on the actuation element 3312 because the anchors 208 open and close due to movement of the actuation element 3412. The force measurement device 3410a is configured to measure the force F1 on the actuation element 3412, and the measurement of the force F1 allows a user to determine if the corresponding tension force provided to the leaflets is at a desired or undesired level.

In the illustrated example, the force measurement device 3410a includes a strain gauge having one or more wires 3416a that extend to a device (not shown) for measuring electrical resistance (e.g., an ohmmeter). After the valve repair device 3400 is attached to the native valve and the tension force provided to the leaflets is determined to be at a desired level, the actuation element 3412 is detached from the valve repair device 3400 and removed from the subject with the delivery system. In other examples, the force measurement device 3410a can be attached to a portion of the actuation element 3412 that is integral to the valve repair device 3400. In these examples, the wire 3416a of the strain gauge can include a detachable and/or weakened portion (e.g., similar to the weakened portions 3414b, 3414c of the wires 3416b, 3416c of the force measurement devices 3410b, 3410c) that allows for the wire 3416a to be separated and/or broken such that the delivery system can be removed from the subject while the strain gauge remains on the valve repair device 3400 that is implanted in the subject.

In some examples, a force measurement device 3410b can be placed on one clasp 330 of the valve repair device 3400 and another force measurement device 3410c can be place on another clasp 330 of the valve repair device 3400. While the illustrated example shows a valve repair device 3400 having two clasps 330, it should be understood that the valve repair device 3400 can have any suitable number of clasps, and it should be understood that a force measurement device can be disposed on one or more of the clasps. In the illustrated example, the force measurement devices 3410b, 3410c are disposed on movable arms 334 of the clasps 330, but it should be understood that the force measurement devices 3410b, 3410c can be positioned at any location on the clasps 330 that allows for the force measurement devices 3410b, 3410c to measure the tension force provided to the leaflets and/or that allows a force, position, or other measured property to be correlated to the tensile force on the leaflets. When the valve repair device 3400 is attached to the leaflets and in the closed position, this attachment between the valve repair device 3400 and the leaflets can cause a tension force on the leaflets and a corresponding force F2 to the clasps 330.

In some implementations, the force measurement devices 3410b, 3410c are configured to measure the force F2 on the corresponding gripping members, and the measurement of the force F2 allows a user to determine if the tension force provided to one or more of the leaflets is at a desired or undesired level.

In some implementations, each of force measurement devices 3410b, 3410c can include a strain gauge that has one or more corresponding wires 3416b, 3416c that extend to a device for measuring electrical resistance.

In some implementations, the wires 3416b, 3416c can each have a detachable and/or weakened portion 3414b, 3414c that allows for the wires to be separated and/or broken such that the strain gauges can remain on the valve repair device 3400, and such that at least a portion of each of the wires 3416b, 3416c can be removed with the delivery system from the subject. In some implementations, the weakened portions 3414b, 3414c can be, for example, a soldered joint, a thinner portion of the wire, a thin portion of a printed circuit trace, a temporary mechanical connection, such as a twisted wire connection, an electrical connector, such as a plug and/or socket, or any other suitable means of allowing for a user to separate and/or break the wires 3416b, 3416c after implantation of the valve repair device 3400. In some examples, a cutting device (not shown) may be used to cut the wires 3416b, 3416c to break the wires.

While the force measurement devices 3410a-3410c are described as including a strain gauge, it should be understood that the force measurement devices 3410a-3410c can take any other suitable form that is capable of determining a tension force applied to the leaflets by the valve repair device 3400. For example, the force measurement devices 3410a-3410c can include a load cell sensor, a strainpad, a piezoelectric sensor, any suitable positional sensor for detecting change in a position of the sensor due to forces created by the connection between the valve repair device and the native valve, or any other suitable type of sensor that is capable of allowing a user to determine a tension force provided to one or more leaflets of the native valve.

While the illustrated example shown in FIG. 34 shows the valve repair device 3400 and the delivery system having three force measurement devices 3410a-3410c, it should be understood that the valve repair device 3400 and/or the delivery system can have any suitable number of force measurement devices. In some instances, only the valve repair device 3400 includes one or more force measurement devices. In other instances, only the delivery device includes one or more force measurement devices.

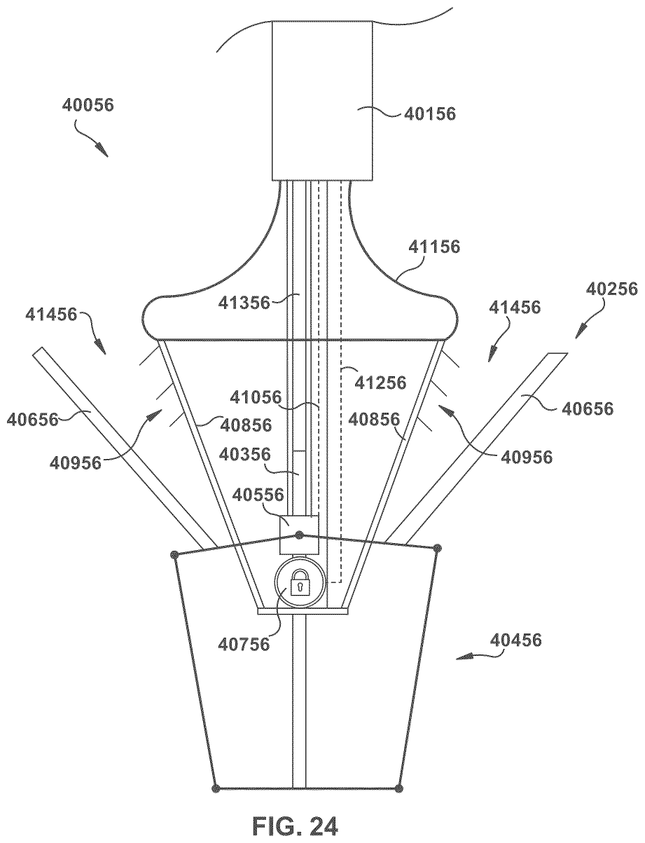

FIG. 24 illustrates another example of one of the many valve repair systems 40056 for repairing a native valve of a subject that the concepts of the present application can be applied to. The valve repair system 40056 includes a delivery device 40156 and a valve repair device 40256.

The valve repair device 40256 includes a base assembly 40456, a pair of paddles 40656, and a pair of gripping members 40856 (e.g., clasp, clip, arm, etc.). In one example, the paddles 40656 can be integrally formed with the base assembly. For example, the paddles 40656 can be formed as extensions of links of the base assembly. In the illustrated example, the base assembly 40456 of the valve repair device 40256 has a shaft 40356, a coupler 40556 configured to move along the shaft, and a lock 40756 configured to lock the coupler in a stationary position on the shaft.

In some implementations, the coupler 40556 is mechanically connected to the paddles 40656, such that movement of the coupler 40556 along the shaft 40356 causes the paddles to move between an open position and a closed position. In this way, the coupler 40556 serves as a means for mechanically coupling the paddles 40656 to the shaft 40356 and, when moving along the shaft 40356, for causing the paddles 40656 to move between their open and closed positions.

In some implementations, the gripping members 40856 are pivotally connected to the base assembly 40456 (e.g., the gripping members 40856 can be pivotally connected to the shaft 40356, or any other suitable member of the base assembly), such that the gripping members can be moved to adjust the width of the opening 41456 between the paddles 40656 and the gripping members 40856. In some implementations, the gripping member 40856 can include an optionally barbed portion 40956 for attaching the gripping members to valve tissue when the valve repair device 40256 is attached to the valve tissue.

In some implementations, when the paddles 40656 are in the closed position, the paddles engage the gripping members 40856, such that, when valve tissue is attached to the barbed portion 40956 of the gripping members, the paddles secure the valve repair device 40256 to the valve tissue. In some implementations, the gripping members 40856 are configured to engage the paddles 40656 such that the barbed portion 40956 engages the valve tissue and the paddles 40656 to secure the valve repair device 40256 to the valve tissue. For example, in certain situations, it can be advantageous to have the paddles 40656 maintain an open position and have the gripping members 40856 move outward toward the paddles 40656 to engage valve tissue and the paddles 40656.

While the example shown in FIG. 24 illustrates a pair of paddles 40656 and a pair of gripping members 40856, it should be understood that the valve repair device 40256 can include any suitable number of paddles and gripping members.

In some implementations, the valve repair system 40056 includes a placement shaft 41356 that is removably attached to the shaft 40356 of the base assembly 40456 of the valve repair device 40256. After the valve repair device 40256 is secured to valve tissue, the placement shaft 41356 is removed from the shaft 40356 to remove the valve repair device 40256 from the remainder of the valve repair system 40056, such that the valve repair device 40256 can remain attached to the valve tissue, and the delivery device 40156 can be removed from a subject's body.

In some implementations, the valve repair system 40056 can also include a paddle control mechanism 41056 (e.g., relatively movable tube(s), shaft(s), etc.), a gripper control mechanism 41156 (e.g., wire(s), line(s), suture(s), etc.), and a lock control mechanism 41256 (e.g., relatively movable tube(s), shaft(s), wire(s), line(s), suture(s), etc.). In some implementations, the paddle control mechanism 41056 is mechanically attached to the coupler 40556 to move the coupler along the shaft, which causes the paddles 40656 to move between the open and closed positions.

The paddle control mechanism 41056 can take any suitable form, such as, for example, a shaft or rod. For example, the paddle control mechanism can comprise a hollow shaft, a catheter tube or a sleeve that fits over the placement shaft 41356 and the shaft 40356 and is connected to the coupler 40556.

In some implementations, the gripper control mechanism 41156 is configured to move the gripping members 40856 such that the width of the opening 41456 between the gripping members and the paddles 40656 can be altered. The gripper control mechanism 41156 can take any suitable form, such as, for example, a line, a suture or wire, a rod, a catheter, etc.

In some implementations, the lock control mechanism 41256 is configured to lock and unlock the lock. In some implementations, the lock 40756 locks the coupler 40556 in a stationary position with respect to the shaft 40356 and can take a wide variety of different forms and the type of lock control mechanism 41256 can be dictated by the type of lock used.

In examples in which the lock 40756 includes a pivotable plate, the lock control mechanism 41256 is configured to engage the pivotable plate to move the plate between the tilted and substantially non-tilted positions. In some implementations, the lock control mechanism 41256 can be, for example, a rod, a suture, a wire, or any other member that is capable of moving a pivotable plate of the lock 40756 between a tilted and substantially non-tilted position.

In some implementations, the valve repair device 40256 is movable from an open position to a closed position. In some implementations, the base assembly 40456 includes links that are moved by the coupler 40556. In some implementations, the coupler 40556 is movably attached to the shaft 40356. In order to move the valve repair device from the open position to the closed position, the coupler 40556 is moved along the shaft 40356, which moves the links.

In some implementations, the gripper control mechanism 41156 is moves the gripping members 40856 to provide a wider or a narrower gap at the opening 41456 between the gripping members and the paddles 40656. In the illustrated example, the gripper control mechanism 41156 includes a line, such as a suture, a wire, etc. that is connected to an opening in an end of the gripper members 40856. In some implementations, when the line(s) is pulled, the gripping members 40856 move inward, which causes the opening 41456 between the gripping members and the paddles 40656 to become wider.

In some implementations, in order to move the valve repair device 40256 from the open position to the closed position, the lock 40756 is moved to an unlocked condition by the lock control mechanism 41256. Once the lock 40756 is in the unlocked condition, the coupler 40556 can be moved along the shaft 40356 by the paddle control mechanism 41056.

In some implementations, after the paddles 40656 are moved to the closed position, the lock 40756 is moved to the locked condition by the lock control mechanism 41256 to maintain the valve repair device 40256 in the closed position. In some implementations, after the valve repair device 40256 is maintained in the locked condition by the lock 40756, the valve repair device 40256 is removed from the delivery device 40156 by disconnecting the shaft 40356 from the placement shaft 41356. In addition, the valve repair device 40256 can be disengaged from the paddle control mechanism 41056, the gripper control mechanism 41156, and the lock control mechanism 41256.

Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

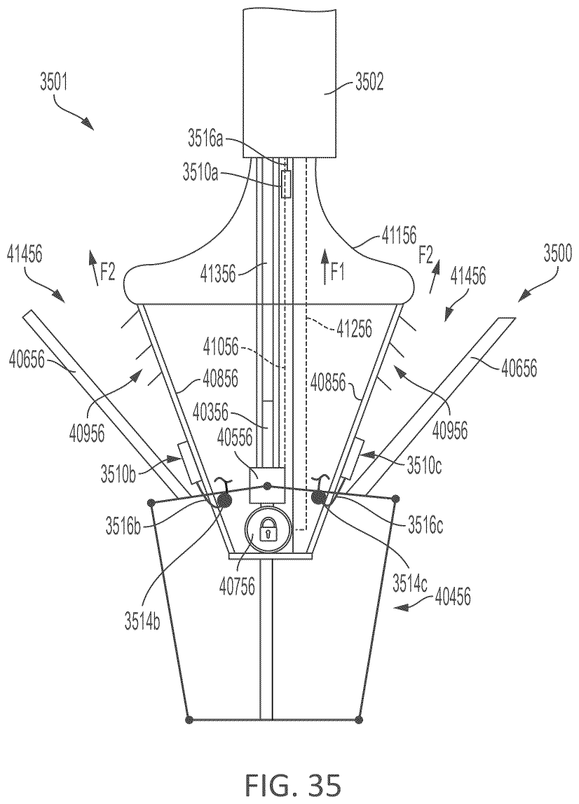

FIG. 35 illustrates another example of one of the many valve repair systems for repairing a native valve of a subject that the concepts of the present application can be applied to, where the valve repair system 3501 is configured to detect a tension force provided to the leaflets due to the connection between a valve repair device 3500 of the system 3501 and the leaflets and/or that is configured to measure a tension force, another force, position, etc. that correlates to tensile force on the leaflets.

In some implementations, the valve repair system 3501 includes a delivery device 3502 and the valve repair device 3500. The delivery device 3502 and valve repair device 3500 can be the same as or similar to the delivery device 40156 and valve repair device 41456, respectively, illustrated by FIG. 24. In some implementations, at least one of the delivery device 3502 and valve repair device 3500 of FIG. 34 includes one or more force measurement devices 3510a-3510c for detecting a tension force provided to the leaflets and/or for detecting a tension force, another force, position, etc. that correlates to tensile force on the leaflets. That is, the connection of the device 3500 with the leaflets causes a tension force to be applied to the leaflets, and the force measurement devices 3510a-3510c are configured to measure the tension force provided to the leaflets or correlated force, distance or other property such that a user can determine if the tension force is outside a preferred tensile force range. For example, if the tension force is below a predetermined amount, there may be a poor attachment between the device 3500 and the leaflets. If the tension force is above a predetermined amount, too much of the leaflets may be grasped by the device 3500.

The force measurement device(s) can be disposed at any location on the device 3500 and/or delivery device 3502 that allows for the force measurement device(s) to measure a tension force provided to the leaflets. In the illustrated example, a force measurement device 3510a is disposed on a paddle control mechanism 41056 of the delivery device 3502. The paddle control mechanism 41056 is mechanically attached to the coupler 40556 to move the coupler 40556 along the shaft 40356, which causes the paddles 40656 to move between the open and closed positions. When the valve repair device 3500 is attached to the leaflets and in the closed position, this attachment between the valve repair device 3500 and the leaflets can cause a tension force on the leaflets and a corresponding force to be applied to the paddles 40656. This force provided to the paddles 40656 can cause the paddles to partially open, which causes a force F1 on the paddle control mechanism 41056 because the opening of the paddles 40656 cause the coupler 40556 to engage the paddle control mechanism 41056. The force measurement device 3510a is configured to measure the force F1 on the paddle control mechanism 41056, and the measurement of the force F1 allows a user to determine if the corresponding tension force provided to the leaflets is at a desired or undesired level.

In the illustrated example, the force measurement device 3510a includes a strain gauge having one or more wires 3516a that extend to a device (not shown) for measuring electrical resistance (e.g., an ohmmeter). After the valve repair device 3500 is attached to the native valve and the tension force provided to the leaflets is determined to be at a desired level, the delivery system 3502 is removed from the subject. In other examples, the force measurement device 3510a may be attached to a portion of the paddle control mechanism 41056 or coupler 40556 that is integral to the valve repair device 3500. In these examples, the wire 3516a of the strain gauge can include a weakened portion (e.g., similar to the weakened portions 3514b, 3514c of the wires 3516b, 3516c of the force measurement devices 3510b, 3510c) that allows for the wire 3516a to be separated and/or broken such that the delivery system can be removed from the subject while the strain gauge remains on the valve repair device 3500 that is implanted in the subject.

In some examples, a force measurement device 3510b can be placed on one gripping member 40856 of the valve repair device 3500 and another force measurement device 3510c can be placed on another gripping member 40856 of the valve repair device 3500. While the illustrated example shows a valve repair device 3500 having two gripping members 40856, it should be understood that the valve repair device 3500 can have any suitable number of gripping members, and it should be understood that a force measurement device can be disposed on one or more of the gripping members.

The force measurement devices 3510b, 3510c can be positioned at any location on the corresponding gripping members 40856 that allow for the force measurement devices 3510b, 3510c to measure the tension force provided to the leaflets and/or that allow a tension force, another force, position, etc. that correlates to tensile force on the leaflets to be measured. When the valve repair device 3500 is attached to the leaflets and in the closed position, this attachment between the valve repair device 3500 and the leaflets can cause a tension force on the leaflets and a corresponding force F2 to the gripping members 40856. The force measurement devices 3510b, 3510c are configured to measure the force F2 on the corresponding gripping members, and the measurement of the force F2 allows a user to determine if the tension force provided to one or more of the leaflets is at a desired or undesired level.

In some implementations, each of force measurement devices 3510b, 3510c can include a strain gauge that has one or more corresponding wires 3516b, 3516c that extend to a device for measuring electrical resistance. In some implementations, the wires 3516b, 3516c can each have a weakened portion 3514b, 3514c that allows for the wires to be separated and/or broken such that the strain gauges can remain on the valve repair device 3500, and such that at least a portion of each of the wires 3516b, 3516c can be removed with the delivery system from the subject. In some implementations, the weakened portions 3514b, 3514c can be, for example, a soldered joint, a thinner portion of the wire, a thin portion of a printed circuit trace, a temporary mechanical connection, such as a twisted wire connection, an electrical connector, such as a plug and/or socket, or any other suitable means of allowing for a user to separate and/or break the wires 3516b, 3516c after implantation of the valve repair device 3500.

In some examples, a cutting device (not shown) may be used to cut the wires 3416b, 3416c to break the wires.

While the force measurement devices 3510a-3510c are described as including a strain gauge, it should be understood that the force measurement devices 3510a-3510c can take any other suitable form that is capable of determining a tension force applied to the leaflets by the valve repair device 3500. For example, the force measurement devices 3510a-3510c can include a load cell sensor, a strainpad, a piezoelectric sensor, any suitable positional sensor for detecting change in a position of the sensor due to forces created by the connection between the valve repair device and the native valve, or any other suitable type of sensor that is capable of allowing a user to determine a tension force provided to one or more leaflets of the native valve.

While the illustrated example shown in FIG. 35 shows the valve repair device 3500 and the delivery system having three force measurement devices 3510a-3510c, it should be understood that the valve repair device 3500 and/or the delivery device 3502 can have any suitable number of force measurement devices. In some instances, only the valve repair device 3500 includes one or more force measurement devices. In other instances, only the delivery device 3502 includes one or more force measurement devices.

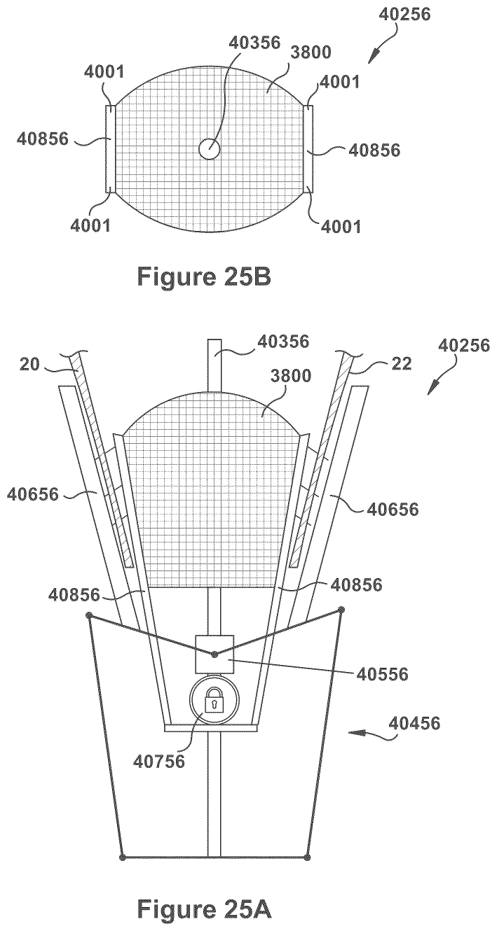

Referring to FIGS. 25A-25B, an example implementation of a valve repair device 40256 has a coaptation element 3800. The valve repair device 40256 can have the same or a similar configuration as the valve repair device illustrated by FIG. 24 with the addition of the coaptation element.

The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 40656 to adjust the size of the coaptation element. In the example illustrated by FIGS. 25A and 25B, the size of the coaptation element 3800 can be reduced by squeezing the coaptation element with the paddles 40656 and can be increased by moving the paddles 40656 away from one another.

In some implementations, the coaptation element 3800 can extend past outer edges 4001 of the gripping members or clasps 40856 as illustrated for providing additional surface area for closing the gap of a mitral valve.

The coaptation element 3800 can be coupled to the valve repair device 40256 in a variety of different ways. For example, the coaptation element 3800 can be fixed to the shaft 40356, can be slidably disposed around the shaft, can be connected to the coupler 40556, can be connected to the lock 40756, and/or can be connected to a central portion of the clasps or gripping members 40856. In some implementations, the coupler 40556 can take the form of the coaptation element 3800. That is, a single element can be used as the coupler 40556 that causes the paddles 40656 to move between the open and closed positions and the coaptation element 3800 that closes the gap between the leaflets 20, 22 when the valve repair device 40256 is attached to the leaflets.

In some implementations, the coaptation element 3800 can be disposed around one or more of the shafts or other control elements of the valve repair system 40056. For example, the coaptation element 3800 can be disposed around the shaft 40356, the shaft 41356, the paddle control mechanism 41056, and/or the lock control mechanism 41256.

The valve repair device 40256 can include any other features for a valve repair device discussed in the present application, and the valve repair device 40256 can be positioned to engage valve tissue as part of any suitable valve repair system (e.g., any valve repair system disclosed in the present application). Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

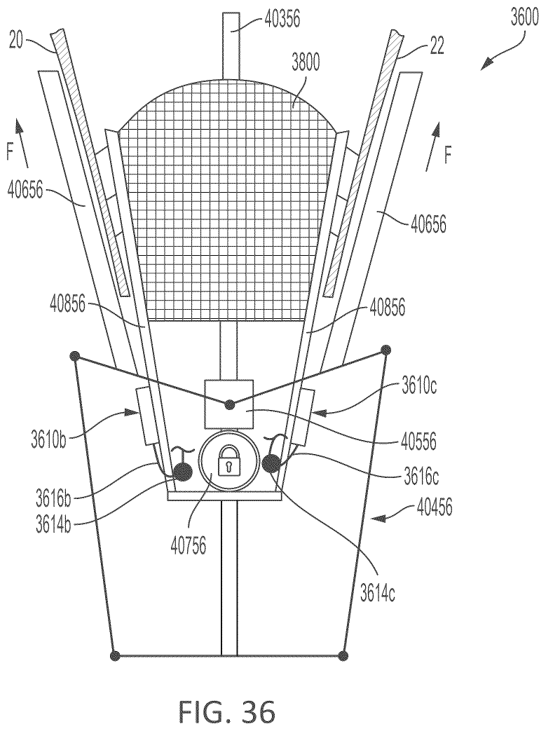

FIG. 36 illustrates another example of one of the many valve repair devices for repairing a native valve of a subject that the concepts of the present application can be applied to, where the valve repair device 3600 is configured to detect a tension force provided to the leaflets and/or to detect a tension force, another force, position, etc. that correlates to tensile force on the leaflets due to the connection between a valve repair device 3600 and the leaflets. The valve repair device 3600 can be the same as or similar to the valve repair device 40256 illustrated by FIGS. 25A-25B.

In some implementations, the valve repair device 3600 of FIG. 36 includes one or more force measurement devices 3610b-3610c for detecting a tension force provided to the leaflets and/or for detecting a tension force, another force, position, etc. that correlates to tensile force on the leaflets. That is, the connection of the device 3600 with the leaflets causes a tension force to be applied to the leaflets, and the force measurement devices 3610b-3610c are configured to measure the tension force provided to the leaflets and/or to measure a tension force, another force, position, etc. that correlates to tensile force on the leaflets such that a user can determine if the tension force is outside a preferred tensile force range. For example, if the tension force is below a predetermined amount, there may be a poor attachment between the device 3600 and the leaflets. If the tension force is above a predetermined amount, too much of the leaflets may be grasped by the device 3600.

The force measurement device(s) can be disposed at any location on the device 3600 that allows for the force measurement device(s) to measure a tension force provided to the leaflets and/or to measure a tension force, another force, position, etc. that correlates to tensile force on the leaflets. For example, a force measurement device 3610b can be placed on one gripping member 40856 of the valve repair device 3600 and another force measurement device 3610c can be placed on another gripping member 40856 of the valve repair device 3500.

While the illustrated example shows a valve repair device 3600 having two gripping members 40856, it should be understood that the valve repair device 3600 can have any suitable number of gripping members, and it should be understood that a force measurement device can be disposed on one or more of the gripping members. The force measurement devices 3610b, 3610c can be positioned at any location on the corresponding gripping members 40856 that allow for the force measurement devices 3610b, 3610c to measure the tension force provided to the leaflets. When the valve repair device 3600 is attached to the leaflets and in the closed position, this attachment between the valve repair device 3600 and the leaflets can cause a tension force on the leaflets and a corresponding force F to the gripping members 40856. The force measurement devices 3610b, 3610c are configured to measure the force on the corresponding gripping members, and the measurement of the force F allows a user to determine if the tension force provided to one or more of the leaflets is at a desired or undesired level.

In some implementations, each of force measurement devices 3610b, 3610c can include a strain gauge that has one or more corresponding wires 3616b, 3616c that extend to a device for measuring electrical resistance. The wires 3616b, 3616c can each have a weakened portion 3614b, 3614c that allows for the wires to be separated and/or broken such that the strain gauges can remain on the valve repair device 3600, and such that at least a portion of each of the wires 3616b, 3616c can be removed with the delivery system from the subject. The weakened portions 3614b, 3614c can be, for example, a soldered joint, a thinner portion of the wire, a thin portion of a printed circuit trace, a temporary mechanical connection, such as a twisted wire connection, an electrical connector, such as a plug and/or socket, or any other suitable means of allowing for a user to break the wires 3616b, 3616c after implantation of the valve repair device 3600. In some examples, a cutting device (not shown) may be used to cut the wires 3616b, 3616c to break the wires.

While the force measurement devices 3610b-3610c are described as including a strain gauge, it should be understood that the force measurement devices 3610b-3510c can take any other suitable form that is capable of determining a tension force applied to the leaflets by the valve repair device 3600. For example, the force measurement devices 3610b-3610c can include a load cell sensor, a strainpad, a piezoelectric sensor, any suitable positional sensor for detecting change in a position of the sensor due to forces created by the connection between the valve repair device and the native valve, or any other suitable type of sensor that is capable of allowing a user to determine a tension force provided to one or more leaflets of the native valve and/or to measure a tension force, another force, position, etc. that correlates to tensile force on the leaflets.

In some implementations, one or more force measurement devices (not shown) can be disposed on a delivery device (not shown) that delivers the valve repair device 3600 to the native valve of the subject. For example, a force measurement device can be attached to a paddle control mechanism (not shown) of the delivery device that is configured to engage the coupler 40556 to move the paddles 40656 between open and closed positions. In some implementations, when the valve repair device 3600 is attached to the leaflets and in the closed position, this attachment between the valve repair device 3600 and the leaflets can cause a tension force on the leaflets and a corresponding force on the paddles 40656. This force provided to the paddles 40656 can cause the paddles to partially open, which causes a force (not shown) on the paddle control mechanism because the opening of the paddles 40656 cause the coupler 40556 to engage the paddle control mechanism.

In some implementations, the force measurement device can be configured to measure the force on the paddle control mechanism, and the measurement of this force allows a user to determine if the corresponding tension force provided to the leaflets is at a desired or undesired level. The force measurement device(s) can take any suitable form, such as, for example, any form for a force measurement device described in the present application.

While the illustrated example shown in FIG. 36 shows the valve repair device 3600 having two force measurement devices 3610b-3610c, it should be understood that the valve repair device 3600 can have any suitable number of force measurement devices. In various instances, both the valve repair device 3600 and a delivery device (not shown) include one or more force measurement devices. In some instances, only the valve repair device 3600 includes one or more force measurement devices. In other instances, only the delivery device includes one or more force measurement devices.

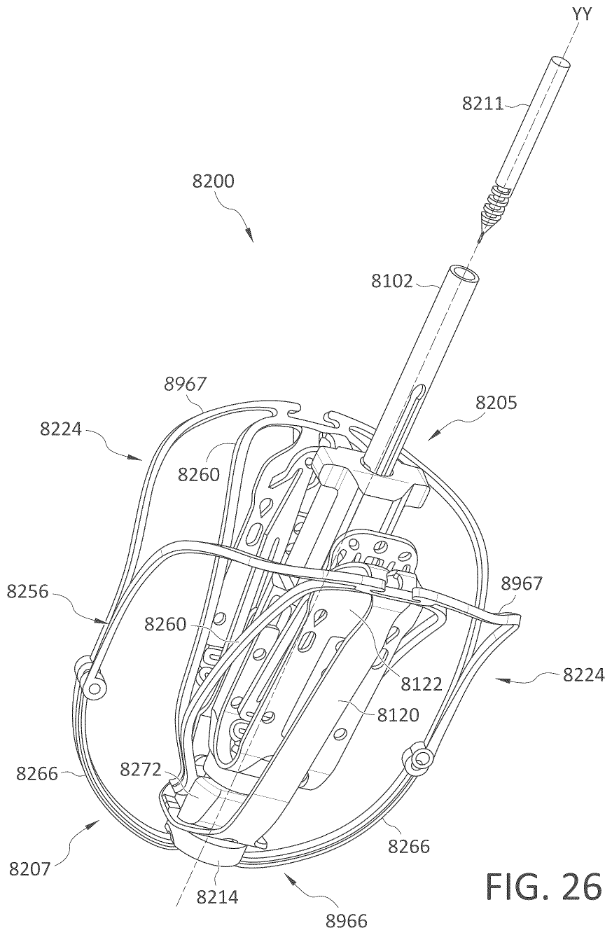

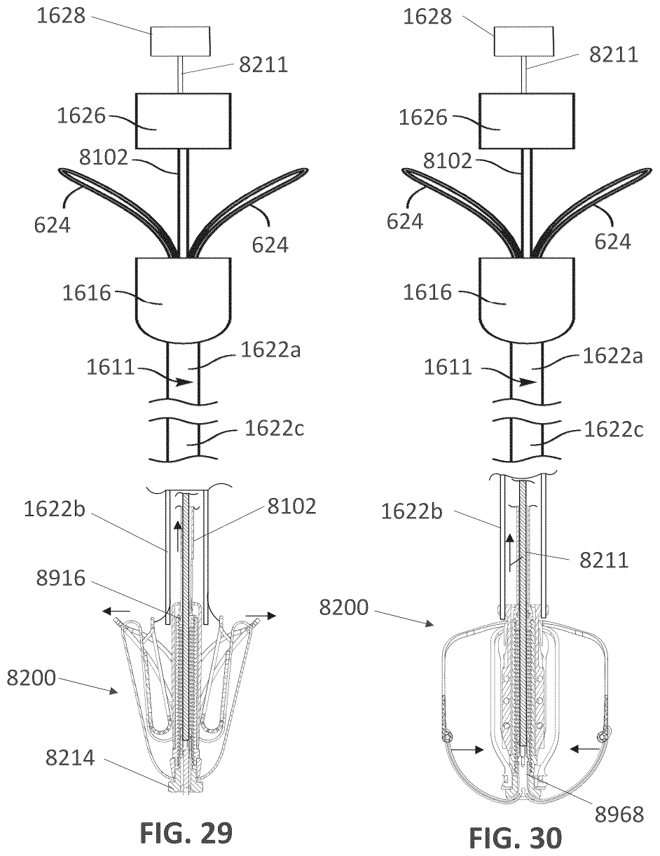

FIGS. 26-30 illustrate another example of one of the many valve repair systems for repairing a native valve of a subject that the concepts of the present application can be applied to. Referring to FIGS. 29 and 30, the valve repair system includes an implant catheter assembly 1611 and an implantable valve repair device 8200. Referring to FIGS. 26-28, the implantable device 8200 includes a proximal or attachment portion 8205, paddles (including inner paddle portions 8122 and outer paddle portions 8120), paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer or adjustable width frame portions 8256 and optional inner frame portions 8260.

In some implementations, a connector 8966 (e.g., shaped metal component, shaped plastic component, tether, wire, strut, line, cord, suture, etc.) attaches to the outer or adjustable width frame portions 8256 at outer ends or connection portions 8266 of the connector 8966 and to a coupler 8972 at an inner end 8968 of the connector 8966 (see FIG. 28). Between the connector 8966 and the attachment portion 8205, the outer or adjustable width frame portions 8256 form a curved shape. In the illustrated example, the shape of the outer or adjustable width frame portions 8256 resembles an apple shape in which the outer or adjustable width frame portions 8256 are wider toward the attachment portion 8205 and narrower toward the distal portion 8207. In some implementations, however, the outer frame portions 8256 can be otherwise shaped.

In the example illustrated in FIG. 26, the outer or adjustable width frame members 8256 (e.g., wire frames, metal frames, etc.) comprise anterior and posterior frame members 8967. The anterior and posterior frame members 8967 are connected to connection portions 8266 of the connector 8966. In the illustrated example, the connector 8966 is attached to both of the adjustable width frame members 8967 via two joints, such as suture joints. In some implementations, however, the connector 8966 can attach to both the anterior and posterior frame members 8967 in any suitable manner.

In some implementations, the inner frame portions 8260 extend from the attachment portion 8205 toward the distal portion 8207. In some implementations, the inner frame portions 8260 then extend inward to form retaining portions 8272 that are attached to the actuation cap 8214. In some implementations, the retaining portions 8272 and the actuation cap 8214 can be configured to attach in any suitable manner.

In some implementations, the optional inner frame portions 8260 are rigid frame portions, while the outer or adjustable width frame portions 8256 are flexible frame portions. The proximal end of the outer frame portions 8256 can connect to or be integrally formed with the proximal end of the inner frame portions 8260, as illustrated in FIG. 26.

In some implementations, the width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214.

In some implementations, the actuation element 8102 is configured to move the inner frame portions 8260 to open and close the paddles in accordance with some implementations disclosed herein.

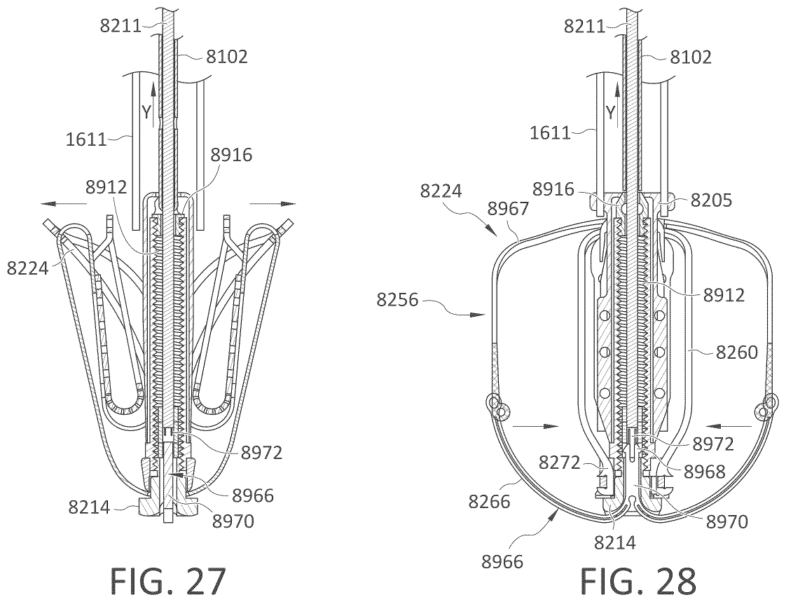

As shown in FIGS. 27 and 28, the connector 8966 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position.

In the illustrated example, the inner end 8968 includes a post 8970 that attaches the outer portions 8266 of the connector 8966 to a coupler 8972. In some implementations, the coupler 8972 is configured to attach and detach from both the width adjustment element 8211 and the receiver 8912.

The coupler 8972 can take a wide variety of different forms. For example, the coupler 8972 can include one or more of a threaded connection, features that mate with threads, detent connections, such as outwardly biased arms, walls, or other portions.

When the coupler 8972 is attached to the width adjustment element 8211, the coupler is released from the receiver 8912. When the coupler 8972 is detached from the width adjustment element 8211, the coupler is secured to the receiver 8912. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used.

The coupler can be configured in a variety of ways as well and can be a separate component or be integral with another portion of the device, e.g., of the connector or inner end of the connector.

In some implementations, the width adjustment element 8211 allows a user to expand or contract the outer frame portions 8256 of the implantable device 8200. In the example illustrated in FIGS. 27 and 28, the width adjustment element 8211 includes an externally threaded end that is threaded into the coupler 8972. In some implementations, the width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as some implementations disclosed above.

In the illustrated example, a driver head 8916 is disposed at a proximal end of the actuation element 8102. In some implementations, the driver head 8916 releasably couples the actuation element 8102 to the receiver 8912. In the illustrated example, the width adjustment element 8211 extends through the actuation element 8102. In some implementations, the actuation element is axially advanced in the direction opposite to direction Y to move the distal cap 8214.

In some implementations, movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. In some implementations, the movement of the outer frame portions 8256 to the narrowed position can allow the device or implant 8200 to maneuver more easily into position for implantation in the heart by reducing the contact and/or friction between the native structures of the heart—e.g., chordae—and the device 8200. In some implementations, the movement of the outer frame portions 8256 to the expanded position provides the anchor portion of the device or implant 8200 with a larger surface area to engage and capture leaflet(s) of a native heart valve.

Referring to FIGS. 29 and 30, an example of an implant catheter assembly 1611 in which clasp actuation lines 624 extend through a handle 1616, the actuation element 8102 is coupled to a paddle actuation control 1626, and the width adjustment element 8211 is coupled to a paddle width control 1628. In some implementations, a proximal end portion 1622a of the shaft or catheter of the implant catheter assembly 1611 can be coupled to the handle 1616, and a distal end portion 1622b of the shaft or catheter can be coupled to the implantable device 8200.

In some implementations, the actuation element 8102 can extend distally from the paddle actuation control 1626, through the handle 1616, through the delivery shaft or catheter of the implant catheter assembly 1611, and through the proximal end of the device 8200, where it couples with the driver head 8916. In some implementations, the actuation element 8102 can be axially movable relative to the outer shaft of the implant catheter assembly 1611 and the handle 1616 to open and close the device.

In some implementations, the width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. In some implementations, the width adjustment element 8211 can be axially movable relative to the actuation element 8102, the outer shaft of the implant catheter assembly 1611, and the handle 1616.

In some implementations, the clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the implant catheter assembly 1611 is an elongate shaft extending axially between the proximal end portion 1622a, which is coupled to the handle 1616, and the distal end portion 1622b, which is coupled to the device 8200. The outer shaft of the implant catheter assembly 1611 can also include an intermediate portion 1622c disposed between the proximal and distal end portions 1622a, 1622b.

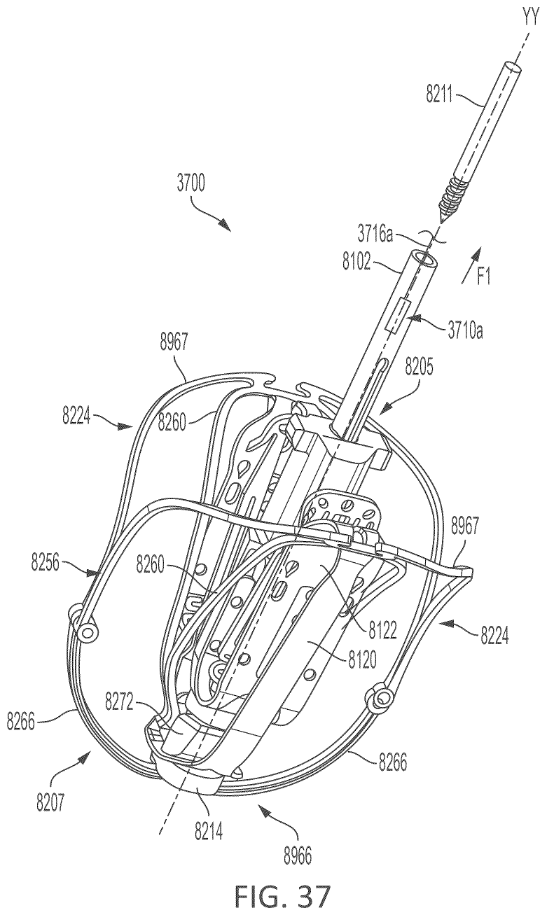

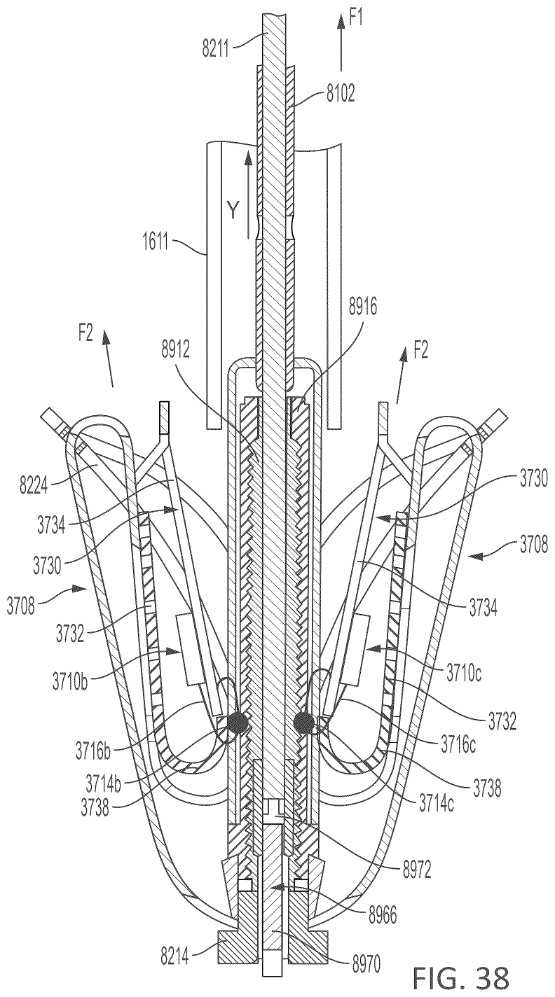

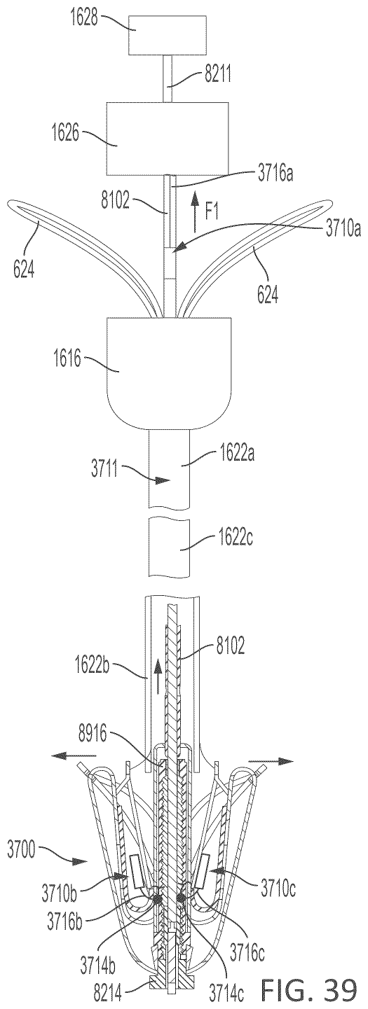

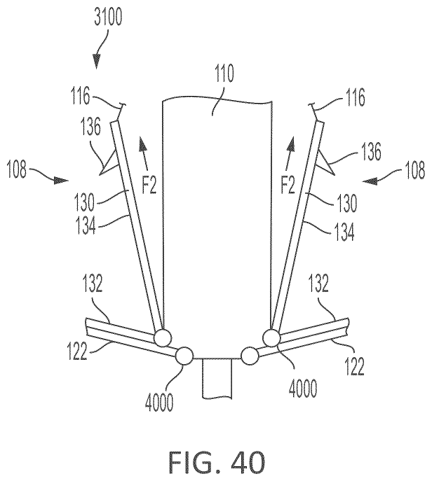

FIGS. 37-39 illustrates another example of one of the many valve repair systems for repairing a native valve of a subject that the concepts of the present application can be applied to, where the valve repair system is configured to detect a tension force provided to the leaflets due to the connection between a valve repair device 3700 and the leaflets. Referring to FIG. 39, the valve repair system includes an implant catheter assembly 3711 and an implantable valve repair device 3700. The implant catheter assembly 3711 and valve repair device 3700 can be the same as or similar to the implant catheter assembly 1611 and valve repair device 8200, respectively, illustrated by FIGS. 26-30.

In some implementations, at least one of the implant catheter assembly 3711 and valve repair device 3700 of FIGS. 37-39 includes one or more force measurement devices 3710a-3710c for detecting a tension force provided to the leaflets and/or to measure a tension force, another force, position, etc. that correlates to tensile force on the leaflets. That is, the connection of the device 3700 with the leaflets causes a tension force to be applied to the leaflets, and the force measurement devices 3710a-3710c are configured to measure the tension force provided to the leaflets and/or to measure a tension force, another force, position, etc. that correlates to tensile force on the leaflets such that a user can determine if the tension force is outside a preferred range. For example, if the tension force is below a predetermined amount, there may be a poor attachment between the device 3700 and the leaflets. If the tension force is above a predetermined amount, too much of the leaflets may be grasped by the device 3700.

The force measurement device(s) can be disposed at any location on the device 3700 and/or implant catheter assembly 3711 that allows for the force measurement device(s) to measure a tension force provided to the leaflets and/or that allows measurement of a tension force, another force, position, etc. that correlates to tensile force on the leaflets. For example, a force measurement device 3710a can be disposed on an actuation element 8102 of the implant catheter assembly 3711.

In some implementations, actuation of the actuation element 8102 opens and closes the one or more paddles 3708 of the device 3700 to grasp the native valve leaflets during implantation. For example, extension of the actuation element 8102 causes a cap 8214 to move distally away from the attachment portion 8205, which moves one or more paddles 3708 to the open position, and retraction of the actuation element 8102 causes the cap to move toward the attachment portion 8205, which moves the one or more paddles 3708 to the closed position.

When the valve repair device 3700 is attached to the leaflets and in the closed position, this attachment between the valve repair device 3700 and the leaflets can cause a tension force on the leaflets and a corresponding force on the one or more paddles 3708. This force provided to the paddles 3708 can cause the paddles to partially open, which causes a force F1 on the actuation element 8102 because the paddles open and close due to movement of the actuation element 8102.

In some implementations, the force measurement device 3710a is configured to measure the force F1 on the actuation element 8102, and the measurement of the force F1 allows a user to determine if the corresponding tension force provided to the leaflets is at a desired or undesired level.

The paddles 3708 can take any suitable form, such as, for example, any form for paddles of a valve repair device described in the present application.

In the illustrated example, the force measurement device 3710a includes a strain gauge having one or more wires 3716a that extend to a device (not shown) for measuring electrical resistance (e.g., an ohmmeter). After the valve repair device 3700 is attached to the native valve and the tension force provided to the leaflets is determined to be at a desired level, the implant catheter assembly 3711 is removed from the subject. In some implementations, the force measurement device 3710a can be attached to a portion of the actuation element 8102 that is integral to the valve repair device 3700. In these examples, the wire 3716a of the strain gauge can include a weakened portion (e.g., similar to the weakened portions 3714b, 3714c of the wires 3716b, 3716c of the force measurement devices 3710b, 3710c) that allows for the wire 3716a to be detached, separated, and/or broken such that the delivery system can be removed from the subject while the strain gauge remains on the valve repair device 3700 that is implanted in the subject.

In some examples, a force measurement device 3710b can be placed on one gripping member 3730 of the valve repair device 3700 and another force measurement device 3710c can be placed on another gripping member 3730 of the valve repair device 3700.

The gripping members 3730 can take any suitable form, such as, for example, any form described in the present application. In the illustrated example, the gripping members 3730 are clasps that include a fixed arm 3732 attached to a paddle 3708, a movable arm 3734 for grasping one or more leaflets of the native valve, and a hinged portion 3738 for hingeably attaching the fixed arm 3732 to the movable arm 3734. While the illustrated example shows a valve repair device 3700 having two gripping members 3730, it should be understood that the valve repair device 3700 can have any suitable number of gripping members, and it should be understood that a force measurement device can be disposed on one or more of the gripping members.

In some implementations, the force measurement devices 3710b, 3710c can be positioned at any location on the corresponding gripping members 3730 that allow for the force measurement devices 3710b, 3710c to measure the tension force provided to the leaflets. When the valve repair device 3700 is attached to the leaflets and in the closed position, this attachment between the valve repair device 3700 and the leaflets can cause a tension force on the leaflets and a corresponding force F2 to the gripping members 3730.

In some implementations, the force measurement devices 3710b, 3710c are configured to measure the force F2 on the corresponding gripping members, and the measurement of the force F2 allows a user to determine if the tension force provided to one or more of the leaflets is at a desired or undesired level.

In some implementations, each of force measurement devices 3710b, 3710c can include a strain gauge that has one or more corresponding wires 3716b, 3716c that extend to a device for measuring electrical resistance. In some implementations, the wires 3716b, 3716c can each have a weakened portion 3714b, 3714c that allows for the wires to be disconnected, separated, and/or broken such that the strain gauges can remain on the valve repair device 3700, and such that at least a portion of each of the wires 3716b, 3716c can be removed with the delivery system from the subject. In some implementations, the weakened portions 3714b, 3714c can be, for example, a soldered joint, a thinner portion of the wire, a thin portion of a printed circuit trace, a temporary mechanical connection, such as a twisted wire connection, an electrical connector, such as a plug and/or socket, or any other suitable means of allowing for a user to break the wires 3716b, 3716c after implantation of the valve repair device 3700.

In some examples, a cutting device (not shown) may be used to cut the wires 3716b, 3716c to break the wires.

While the force measurement devices 3710a-3710c are described as including a strain gauge, it should be understood that the force measurement devices 3710a-3710c can take any other suitable form that is capable of determining a tension force applied to the leaflets by the valve repair device 3700. For example, the force measurement devices 3710a-3710c can include a load cell sensor, a strainpad, a piezoelectric sensor, any suitable positional sensor for detecting change in a position of the sensor due to forces created by the connection between the valve repair device and the native valve, or any other suitable type of sensor that is capable of allowing a user to determine a tension force provided to one or more leaflets of the native valve. and/or that is configured to allow a user to determine a tension force, another force, position, etc. that correlates to tensile force on the leaflets.

While the illustrated example shown in FIGS. 37-39 shows the valve repair device 3700 and the implant catheter assembly 3711 having three force measurement devices 3710a-3710c, it should be understood that the valve repair device 3700 and/or the implant catheter assembly 3711 can have any suitable number of force measurement devices. In some instances, only the valve repair device 3700 includes one or more force measurement devices. In other instances, only the implant catheter assembly 3711 includes one or more force measurement devices.

An example method of determining if a tension force provided to the leaflets due to the connection between a valve repair device and the leaflets is at a desired level includes delivering a valve repair device to a native valve of a subject with a delivery device or implant catheter assembly. The valve repair device and delivery device or implant catheter assembly can take any suitable form, such as, for example, any form described in the present application where at least one of the valve repair device and delivery device or implant catheter assembly include one or more force measurement devices for detecting the tension force applied to the leaflets. The force measurement device can take any suitable form, such as, for example, any form described in the present application.

In some implementations, the method further includes attaching the valve repair device to one or more leaflets of the native valve. In some implementations, after the valve repair device is attached to the one or more leaflets, the method includes determining the tension force provided to the leaflets based on a force measurement detected by the force measurement device.

In some implementations, the method can include removing the delivery device or implant catheter assembly from the subject if the tension force provided to the leaflets is at a desired level.

In some implementations, the method can include detaching the valve repair device from one or more leaflets if the tension force provided to the leaflets is at an undesired level and reattaching the valve repair device to the leaflets.

In some implementations, the method can include detaching and reattaching the valve repair device to the leaflets until the tension force provided to the leaflets is at a desired level.

In some implementations, a force measurement device can be integrally formed with one or more of the components of the valve repair device.

The integrally formed force measurement device can take a variety of different forms. For example, the integrally formed force measurement device can comprise a material that emits a signal when the material is stretched or strained. For example, the material can be a piezoelectric material that produces a voltage in response to strain applied to the piezoelectric material. For example, the piezoelectric material can be a ceramic piezoelectric material. In some implementations, the piezoelectric material produces a voltage when subjected to a strain, at a known ratio of strain to voltage. However, the integrally formed force measurement device can comprise any material that produces a signal in response to an applied strain.

In some implementations, the material that forms the integral force measurement device can be a material that is different than a remainder of the component that includes the integral force measurement device. For example, the component that includes the integral force measurement device can be made from a metal, such as steel, aluminum, a shape memory alloy, such as nitinol, with the integrally formed force measurement device being made from a ceramic material. In some implementations, the material that forms the integral force measurement device can be the same as the material that forms the remainder of the component that includes the integral force measurement device. When the integrally formed force measurement device is formed from the same material as the remainder of the component, the integrally formed force measurement device can be configured (e.g., by changing the size and or shape of the integrally formed force measurement device as compared to the remainder of the device) to cause the integrally formed force measurement device to provide a signal in response to strain. For example, the integrally formed force measurement device can be a portion of the component that is narrower and or thinner than the remainder of the component to provide a signal in response to an applied strain.

A variety of the components of the valve repair device can include and integral force measurement device. For example, any of the moving joints of a valve repair device can be configured as an integral force measurement device. In some implementations, one or more of the moving joints of the valve repair device can be configured to provide a signal, such as a voltage signal, in response to the application of strain to the moving joint. For example, the hinge portion of one or more clasp and/or one or more of the pivoting or flexing portions of the paddles or paddle frames can be configured to provide a signal, such as a voltage signal, in response to the application of strain. The signal, such as a voltage signal, can have a known ratio to strain.

In some implementations, the integral force measurement device can be configured to transmit the strain indicating signal without being physically attached or connected to a device that correlates the strain indicating signal to the strain. For example, one or more components of the delivery system can be configured to read the strain indicating signal without physically attaching to the integral force measurement device. For example, one or more of the actuation element 112, the actuation lines 116, a component of an implant catheter assembly 1611, such as the coupler and or the catheter, and the steerable catheter can be configured to read the strain indicating signal without physically attaching to the integral force measurement device.

In some implementations, one or more of the actuation element 112, the actuation lines 116, a component of an implant catheter assembly 1611, such as the coupler and or the catheter, and the steerable catheter can have an induction-based measurement system that reads the electrical signal, such as a voltage signal from a piezoelectric material, without physically attaching to the integral force measurement device.

In some implementations, the induction-based measurement system can read the strain indicating signal, such as a change in potential, as the valve repair device is manipulated, to indicate whether the strain on the leaflet has exceeded a predetermined acceptable strain and or to determine how much leaflet is captured. In some implementations, since the measurement system works on induction reading a current that the implant components generate passively, disconnection does not require a separate mechanical disconnection from the integral force measurement device.

In some implementations, the force measurement device stays with the device after implantation and removal of the device from the delivery system. In some implementations, the force measurement device and/or a monitoring system can be configured to transmit, read, and/or analyze the strain indication signal. For example, the device can include a processor or microchip and/or a power source and the monitoring system can include a wireless communication device. In some implementations, the processor or microchip and/or power source can store and/or transmit data and a wireless communication device can read the data on the force measurement device(s) of the valve repair device non-invasively. For example, the wireless communication device can read the data on the force measurement device(s) of the valve repair device from outside the subject's body, allowing continued data from the force measurement device to be read and analyzed.

In some implementations, the wireless communication device is omitted and the data from the force measurement device(s) can be read in a minimally invasive way. For example, a minimally invasive guide wire can be routed through the subject's vasculature back to or near the valve repair device to read the amount of strain sensed by the force measurement device and/or to retrieve data stored by the processor or microchip.

Referring to FIG. 40, in some instances, an integral force measurement device 4000 can be part of a hinge or joint of the clasp 130 and/or can be a part of a joint of the paddle 122. While the illustrated example shows a valve repair device 3100 having two gripping members, it should be understood that the valve repair device 3100 can have any suitable number of gripping members, and it should be understood that an integral force measurement device can be disposed on one or more of the gripping members. While the illustrated example shows a valve repair device 3100 having two paddles, it should be understood that the valve repair device 3100 can have any suitable number of paddles, and it should be understood that an integral force measurement device can be disposed on one or more of the paddles. In the illustrated example, the integral force measurement devices 4000 are disposed on the hinge or joint of the clasps 130 of the gripping members and/or the hinge or joint of the paddles, but it should be understood that the integral force measurement devices 4000 can be provided at any location on the gripping members and/or the paddles that allows for the integral force measurement devices to measure the tension force provided to the leaflets.

When the valve repair device 3100 is attached to the leaflets and in the closed position, this attachment between the valve repair device 3100 and the leaflets can cause a tension force on the leaflets and a corresponding force to the gripping members. In some implementations, the force measurement device(s) 4000 are configured to measure the force F2 on the corresponding gripping members, and the measurement of the force F2 allows a user to determine if the corresponding tension force provided to one or more of the leaflets is at a desired or undesired level.

While the illustrated example shown in FIG. 40 shows the valve repair device 3100 having four force measurement devices 4000, it should be understood that the valve repair device 3100 can have any suitable number of force measurement devices.

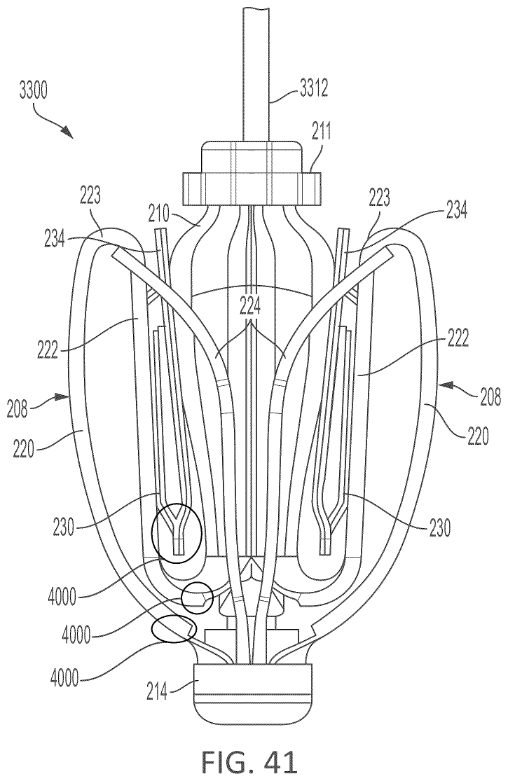

FIG. 41 illustrates an example of a device 3300 that is configured to detect a tension force provided to the leaflets due to the connection between the device 3300 and the leaflets. The device 3300 is similar to the device 200 illustrated by FIG. 22, except the device 3300 of FIG. 33 includes one or more integral force measurement devices 4000 for detecting a tension force provided to the leaflets. That is, the connection of the device 3300 with the leaflets causes a tension force on the leaflets, and the integral force measurement devices 4000 are configured to measure the tension force provided to the leaflets such that a user can determine if the tension force is higher or lower than an optimal force range. For example, if the tension force is below a predetermined amount, there may be a poor attachment between the device 3300 and the leaflets. If the tension force is above a predetermined amount, too much of the leaflets may be grasped by the device 3300.

The force measurement device(s) can be disposed at any location on the device 3300 that allows for the force measurement device(s) to measure a tension force provided to the leaflets and/or to measure a tension force, another force, position, etc. that correlates to tensile force on the leaflets. In the example illustrated by FIG. 41 a force measurement device 4000 can be disposed on a joint or hinge of the gripping member or clasp, can be disposed on a joint or hinge of the inner paddle 222, and/or can be disposed on a joint or hinge of the outer paddle 220.

In some implementations, actuation of the actuation element 3312 opens and closes the one or more anchors 208 of the device 3300 to grasp the native valve leaflets during implantation. For example, extension of the actuation element 3312 can cause the cap 214 of the device 3300 to move away from the coaptation element 210, which moves one or more anchors 208 to the open position, and retraction of the actuation element 3312 can cause the cap 214 to move toward the coaptation element 210, which moves the one or more anchors 208 to the closed position.

When the valve repair device 3300 is attached to the leaflets and in the closed position, this attachment between the valve repair device 3300 and the leaflets can cause a tension force on the leaflets and a corresponding force on the one or more anchors 208. This force provided to the anchors 208 may cause the anchors to partially open.

In some implementations, after the valve repair device 3300 is attached to the native valve and the tension force provided to the leaflets is determined to be at a desired level, the actuation element 3312 is detached from the valve repair device 3300 and removed from the subject with the delivery system.

In some examples, an integral force measurement device 4000 can be included on one or both clasps 230 of the valve repair device 3300. While the illustrated example shows a valve repair device 3300 having two clasps 230, it should be understood that the valve repair device 3300 can have any suitable number of clasps, and it should be understood that an integral force measurement device can be disposed on one or more of the clasps. In the illustrated example, the integral force measurement devices are disposed on the hinge or joint of the clasps 230, but it should be understood that the integral force measurement devices 4000 can be positioned at any location on the clasps 230 that allows for the force measurement devices 4000 to measure the tension force provided to the leaflets.

In some examples, an integral force measurement device 4000 can be included on one or both inner paddles 222 of the valve repair device 3300. While the illustrated example shows a valve repair device 3300 having two inner paddles 222, it should be understood that the valve repair device 3300 can have any suitable number of inner paddles, and it should be understood that an integral force measurement device can be disposed on one or more of the inner paddles. In the illustrated example, the integral force measurement devices are disposed on the hinge or joint of the inner paddles 222, but it should be understood that the integral measurement devices 4000 can be positioned at any location on the inner paddles 222 that allows for the integral force measurement devices 4000 to measure the tension force provided to the leaflets.

In some examples, an integral force measurement device 4000 can be included on one or both outer paddles 220 of the valve repair device 3300. While the illustrated example shows a valve repair device 3300 having two outer paddles 220, it should be understood that the valve repair device 3300 can have any suitable number of inner paddles, and it should be understood that an integral force measurement device 4000 can be disposed on one or more of the outer paddles. In the illustrated example, the integral force measurement devices are disposed on the hinge or joint of the outer paddles 220, but it should be understood that the integral measurement devices 4000 can be positioned at any location on the outer paddles 220 that allows for the force measurement devices 4000 to measure the tension force provided to the leaflets.

When the valve repair device 3300 is attached to the leaflets and in the closed position, this attachment between the valve repair device 3300 and the leaflets can cause a tension force on the leaflets and a corresponding force to the clasps 230. In some implementations, the force measurement devices 4000 are configured to measure the force on the corresponding gripping member(s), inner paddle(s), and/or outer paddle(s) and the measurement of the force allows a user to determine if the tension force provided to one or more of the leaflets is at a desired or undesired level. While the illustrated example shown in FIG. 41 shows the valve repair device 3300 having six integral force measurement devices 4000, it should be understood that the valve repair device 3300 can have any number of integral force measurement devices.

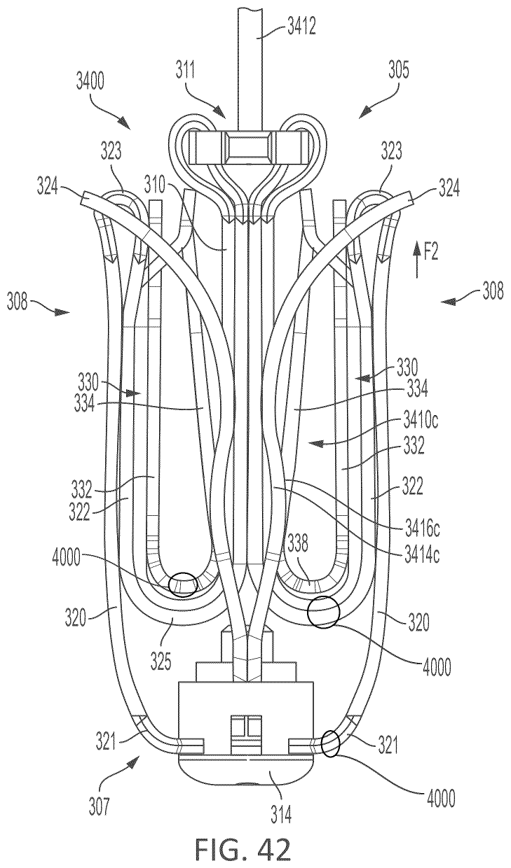

FIG. 42 illustrates an example of a device 3400 and/or an actuation element 3412 of a delivery system that are configured to detect a tension force provided to the leaflets due to the connection between the device 3400 and the leaflets. The device 3400 can be the same as or similar to the device 300 illustrated by FIG. 23.

In some implementations, the device 3400 of FIG. 34 includes one or more integral force measurement devices 4000 for detecting a tension force provided to the leaflets and/or to measure a tension force, another force, position, etc. that correlates to tensile force on the leaflets. That is, the connection of the device 3400 with the leaflets causes a tension force to be applied to the leaflets, and the integral force measurement devices 4000 are configured to measure the tension force provided to the leaflets and/or to measure a force that can be correlated to the tension force applied to the leaflets such that a user can determine if the tension force is higher or lower than an optimal tension force range. For example, if the tension force is below a predetermined amount, there may be a poor attachment between the device 3400 and the leaflets. If the tension force is above a predetermined amount, too much of the leaflets may be grasped by the device 3400.

The integral force measurement device(s) can be disposed at any location on the device 3400 that allows for the force measurement device(s) to measure a tension force provided to the leaflets.

In some implementations, actuation of the actuation element 3412 opens and closes the one or more anchors 308 of the device 3400 to grasp the native valve leaflets during implantation. For example, extension of the actuation element 3412 can cause the cap 314 of the device 3400 to move away from the coaptation element 310, which moves one or more anchors 308 to the open position, and retraction of the actuation element 3412 can cause the cap 314 to move toward the coaptation element 310, which moves the one or more anchors 308 to the closed position. When the valve repair device 3400 is attached to the leaflets and in the closed position, this attachment between the valve repair device 3400 and the leaflets can cause a tension force on the leaflets and a corresponding force on the one or more anchors 308. This force provided to the anchors 308 can cause the anchors to partially open.

In some examples, an integral force measurement device 4000 can be included on one or both clasps 330 of the valve repair device 3400. While the illustrated example shows a valve repair device 3400 having two clasps 330, it should be understood that the valve repair device 3400 can have any suitable number of clasps, and it should be understood that an integral force measurement device can be disposed on one or more of the clasps. In the illustrated example, the integral force measurement devices are disposed on the hinge or joint of the clasps 330, but it should be understood that the integral force measurement devices 4000 can be positioned at any location on the clasps 330 that allows for the force measurement devices 4000 to measure the tension force provided to the leaflets.

In some examples, an integral force measurement device 4000 can be included on one or both inner paddles 322 of the valve repair device 3400. While the illustrated example shows a valve repair device 3400 having two inner paddles 322, it should be understood that the valve repair device 3400 can have any suitable number of inner paddles, and it should be understood that an integral force measurement device can be disposed on one or more of the inner paddles. In the illustrated example, the integral force measurement devices are disposed on the hinge or joint of the inner paddles 322, but it should be understood that the integral measurement devices 4000 can be positioned at any location on the inner paddles 322 that allows for the force measurement devices 4000 to measure the tension force provided to the leaflets.

In some examples, an integral force measurement device 4000 can be included on one or both outer paddles 320 of the valve repair device 3400. While the illustrated example shows a valve repair device 3400 having two outer paddles 320, it should be understood that the valve repair device 3400 can have any suitable number of inner paddles, and it should be understood that an integral force measurement device 4000 can be disposed on one or more of the outer paddles. In the illustrated example, the integral force measurement devices are disposed on the hinge or joint of the outer paddles 320, but it should be understood that the integral measurement devices 4000 can be positioned at any location on the outer paddles 320 that allows for the force measurement devices 4000 to measure the tension force provided to the leaflets.

When the valve repair device 3400 is attached to the leaflets and in the closed position, this attachment between the valve repair device 3400 and the leaflets can cause a tension force on the leaflets and a corresponding force to the clasps 330. The integral force measurement devices 4000 are configured to measure and/or correlate the force on the corresponding gripping members, and the measurement and/or correlation of the force allows a user to determine if the tension force provided to one or more of the leaflets is at a desired or undesired level.

While the illustrated example shown in FIG. 42 shows the valve repair device 3400 having three integral force measurement devices 4000, it should be understood that the valve repair device 3400 and/or the delivery system can have any suitable number of force measurement devices.

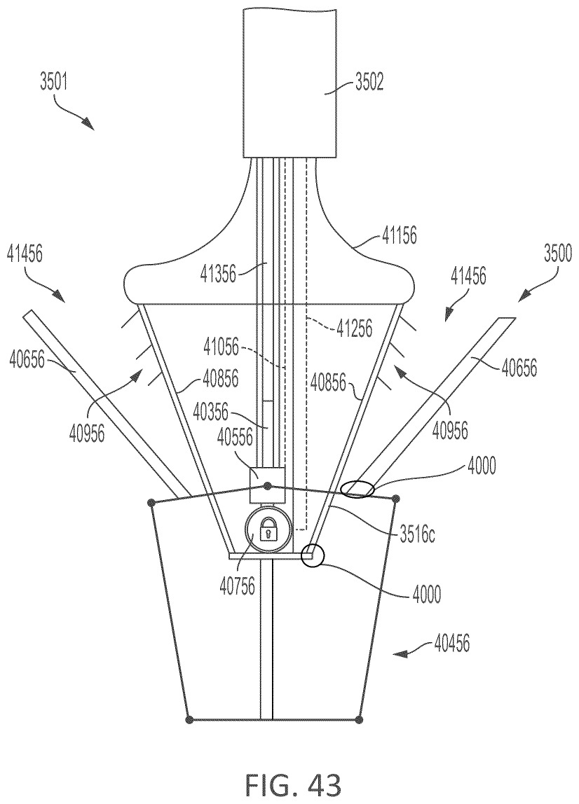

FIG. 43 illustrates another example of one of the many valve repair systems for repairing a native valve of a subject that the concepts of the present application can be applied to, where the valve repair system 3501 is configured to measure a tension force, another force, position, etc. that correlates to tensile force on the leaflets.

In some implementations, the valve repair system 3501 includes a delivery device 3502 and the valve repair device 3500. In some implementations, the delivery device 3502 and valve repair device 3500 are the same as or similar to the delivery device 40156 and valve repair device 41456, respectively, illustrated by FIG. 24.

In some implementations, the valve repair device 3500 of FIG. 43 includes one or more integral force measurement devices 4000 for detecting a tension force provided to the leaflets and/or for detecting a tension force, another force, position, etc. that correlates to tensile force on the leaflets. That is, the connection of the device 3500 with the leaflets causes a tension force to be applied to the leaflets, and the force measurement devices 4000 are configured to measure the tension force provided to the leaflets or correlated force, distance or other property such that a user can determine if the tension force is outside a preferred tensile force range. For example, if the tension force is below a predetermined amount, there may be a poor attachment between the device 3500 and the leaflets. If the tension force is above a predetermined amount, too much of the leaflets may be grasped by the device 3500.

The force measurement device(s) can be disposed at any location on the device 3500 that allows for the force measurement device(s) to measure a tension force provided to the leaflets. A paddle control mechanism 41056 is mechanically attached to the coupler 40556 to move the coupler 40556 along the shaft 40356, which causes the paddles 40656 to move between the open and closed positions. When the valve repair device 3500 is attached to the leaflets and in the closed position, this attachment between the valve repair device 3500 and the leaflets can cause a tension force on the leaflets and a corresponding force to be applied to the paddles 40656. This force provided to the paddles 40656 can cause the paddles to partially open.

In some implementations, after the valve repair device 3500 is attached to the native valve and the tension force provided to the leaflets is determined to be at a desired level, the delivery system 3502 is removed from the subject.

In some examples, an integral force measurement device 4000 can be included on one or both gripping members 40856 of the valve repair device 3500. While the illustrated example shows a valve repair device 3500 having two gripping members 40856, it should be understood that the valve repair device 3500 can have any suitable number of gripping members 40856, and it should be understood that an integral force measurement device can be disposed on one or more of the gripping members 40856. In the illustrated example, the integral force measurement devices are disposed on the hinge or joint of the gripping members 40856, but it should be understood that the integral force measurement devices 4000 can be positioned at any location on the gripping members 40856 that allows for the force measurement devices 4000 to measure the tension force provided to the leaflets.

In some examples, an integral force measurement device 4000 can be included on one or both paddles 40656 of the valve repair device 3500. While the illustrated example shows a valve repair device 3500 having two paddles 40656, it should be understood that the valve repair device 3500 can have any suitable number of paddles, and it should be understood that an integral force measurement device can be disposed on one or more of the paddles. In the illustrated example, the integral force measurement devices are disposed on the hinge or joint of the paddles 40656, but it should be understood that the integral measurement devices 4000 can be positioned at any location on the paddles 40656 that allows for the force measurement devices 4000 to measure the tension force provided to the leaflets.

While the illustrated example shown in FIG. 43 shows the valve repair device 3500 having four force measurement devices 4000, it should be understood that the valve repair device 3500 and/or the delivery device 3502 can have any suitable number of force measurement devices.

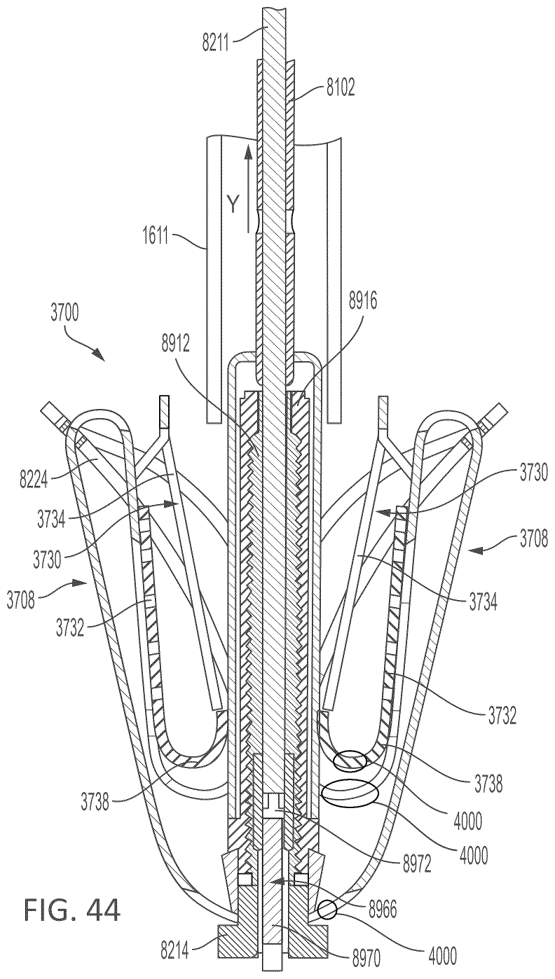

FIG. 44 illustrates another example of one of the many valve repair systems for repairing a native valve of a subject that the concepts of the present application can be applied to, where the valve repair system is configured to detect a tension force provided to the leaflets due to the connection between a valve repair device 3700 and the leaflets. In some implementations, the valve repair system includes an implant catheter assembly 3711 and an implantable valve repair device 3700. The implant catheter assembly 3711 and valve repair device 3700 can be the same as or similar to the implant catheter assembly 1611 and valve repair device 8200, respectively, illustrated by FIGS. 26-30.

In some implementations, the valve repair device 3700 of FIG. 44 includes one or more integral force measurement devices 4000 for detecting a tension force provided to the leaflets and/or to measure a tension force, another force, position, etc. that correlates to tensile force on the leaflets. That is, the connection of the device 3700 with the leaflets causes a tension force to be applied to the leaflets, and the integral force measurement devices 4000 are configured to measure the tension force provided to the leaflets and/or to measure a tension force, another force, position, etc. that correlates to tensile force on the leaflets such that a user can determine if the tension force is outside a preferred range. For example, if the tension force is below a predetermined amount, there may be a poor attachment between the device 3700 and the leaflets. If the tension force is above a predetermined amount, too much of the leaflets may be grasped by the device 3700.

The integral force measurement device(s) 4000 can be disposed at any location on the device 3700 that allows for the force measurement device(s) to measure a tension force provided to the leaflets and/or that allows measurement of a tension force, another force, position, etc. that correlates to tensile force on the leaflets.

In some implementations, actuation of the actuation element 8102 opens and closes the one or more paddles 3708 of the device 3700 to grasp the native valve leaflets during implantation. For example, extension of the actuation element 8102 causes a cap 8214 to move distally away from the attachment portion 8205, which moves one or more paddles 3708 to the open position, and retraction of the actuation element 8102 causes the cap to move toward the attachment portion 8205, which moves the one or more paddles 3708 to the closed position.

When the valve repair device 3700 is attached to the leaflets and in the closed position, this attachment between the valve repair device 3700 and the leaflets can cause a tension force on the leaflets and a corresponding force on the one or more paddles 3708. This force provided to the paddles 3708 may cause the paddles to partially open. The integral force measurement device 4000 is configured to measure the force to allow a user to determine if the corresponding tension force provided to the leaflets is at a desired or undesired level. The paddles 3708 can take any suitable form, such as, for example, any form for paddles of a valve repair device described in the present application.

In some implementations, after the valve repair device 3700 is attached to the native valve and the tension force provided to the leaflets is determined to be at a desired level, the implant catheter assembly 3711 is removed from the subject.

In some examples, an integral force measurement device 4000 can be included on one or both clasps 3730 of the valve repair device 3700. While the illustrated example shows a valve repair device 3700 having two clasps 330, it should be understood that the valve repair device 3700 can have any suitable number of clasps, and it should be understood that an integral force measurement device can be disposed on one or more of the clasps. In the illustrated example, the integral force measurement devices are disposed on the hinge or joint of the clasps 3730, but it should be understood that the integral force measurement devices 4000 can be positioned at any location on the clasps 3730 that allows for the force measurement devices 4000 to measure the tension force provided to the leaflets.

In some examples, an integral force measurement device 4000 can be included on one or both inner paddles 8122 of the valve repair device 3700. While the illustrated example shows a valve repair device 3700 having two inner paddles 8122, it should be understood that the valve repair device 3700 can have any suitable number of inner paddles 8122, and it should be understood that an integral force measurement device can be disposed on one or more of the inner paddles. In the illustrated example, the integral force measurement devices are disposed on the hinge or joint of the inner paddles 8122, but it should be understood that the integral measurement devices 4000 can be positioned at any location on the inner paddles 8122 that allows for the force measurement devices 4000 to measure the tension force provided to the leaflets.

In some examples, an integral force measurement device 4000 can be included on one or both outer paddles 8120 of the valve repair device 3700. While the illustrated example shows a valve repair device 3700 having two outer paddles 8120, it should be understood that the valve repair device 3700 can have any suitable number of inner paddles, and it should be understood that an integral force measurement device 4000 can be disposed on one or more of the outer paddles. In the illustrated example, the integral force measurement devices are disposed on the hinge or joint of the outer paddles 8120, but it should be understood that the integral measurement devices 4000 can be positioned at any location on the outer paddles 8120 that allows for the force measurement devices 4000 to measure the tension force provided to the leaflets.

The gripping members 3730 can take any suitable form, such as, for example, any form described in the present application. In the illustrated example, the gripping members 3730 are clasps that include a fixed arm 3732 attached to a paddle 3708, a movable arm 3734 for grasping one or more leaflets of the native valve, and a hinged portion 3738 for hingeably attaching the fixed arm 3732 to the movable arm 3734. While the illustrated example shows a valve repair device 3700 having two gripping members 3730, it should be understood that the valve repair device 3700 can have any suitable number of gripping members, and it should be understood that a force measurement device can be disposed on one or more of the gripping members. The force measurement devices 4000 can be positioned at any location on the corresponding gripping members 3730 that allow for the force measurement devices 4000 to measure the tension force provided to the leaflets.

When the valve repair device 3700 is attached to the leaflets and in the closed position, this attachment between the valve repair device 3700 and the leaflets can cause a tension force on the leaflets and a corresponding force to the gripping members 3730. The force measurement devices 4000 are configured to measure the force on the corresponding gripping members, and the measurement of the force allows a user to determine if the tension force provided to one or more of the leaflets is at a desired or undesired level.

An example method of determining if a tension force provided to the leaflets due to the connection between a valve repair device and the leaflets is at a desired level includes delivering a valve repair device to a native valve of a subject with a delivery device or implant catheter assembly. The valve repair device and delivery device or implant catheter assembly can take any suitable form, such as, for example, any form described in the present application where the valve repair device includes one or more integral force measurement devices for detecting the tension force applied to the leaflets. The integral force measurement device can take any suitable form, such as, for example, any form described in the present application.

In some implementations, the method further includes attaching the valve repair device to one or more leaflets of the native valve. In some implementations, after the valve repair device is attached to the one or more leaflets, the method includes determining the tension force provided to the leaflets based on a force measurement detected by the integral force measurement device.

In some implementations, the method can include removing the delivery device or implant catheter assembly from the subject if the tension force provided to the leaflets is at a desired level.

In some implementations, the method can include detaching the valve repair device from one or more leaflets if the tension force provided to the leaflets is at an undesired level and reattaching the valve repair device to the leaflets.

In some implementations, the method can include detaching and reattaching the valve repair device to the leaflets until the tension force provided to the leaflets is at a desired level.

### EXAMPLES

Example 1. A valve repair device for repairing a native valve of a heart, the valve repair device comprising:
at least one paddle movable between an open position and a closed position, wherein the at least one paddle is configured to attach the valve repair device to the native valve;
at least one gripping member configured to capture the native valve;
a force measurement device attached to the at least one gripping member; and
wherein the force measurement device is configured to detect a force provided to the at least one gripping member when the valve repair device is attached to the native valve.
Example 2. The valve repair device according to example 1, wherein the at least one gripping member comprises a clasp having:
a fixed arm attached to the at least one paddle;
   a movable arm; and
   a hinge portion hingeably connecting the fixed arm to the movable arm.
Example 3. The valve repair device according to example 2, wherein the force measurement device is attached to the movable arm of the clasp.
Example 4. The valve repair device according to example 2, wherein the force measurement device is attached to the hinge portion of the clasp.
Example 5. The valve repair device according to any one of examples 1-4, wherein the force measurement device comprises a strain gauge.
Example 6. The valve repair device according to example 5, wherein the force measurement device comprises a wire attached to the strain gauge, and wherein the wire comprises a weakened portion that is configured to allow a user to break the wire after attaching the valve repair device to the native valve.
Example 7. The valve repair device according to any one of examples 1-6, wherein the at least one gripping member comprises a first gripping member and a second gripping member, wherein the force measurement device is attached to the first gripping member and a second force measurement device is attached to the second gripping member.
Example 8. The valve repair device according to any one of examples 1-7, further comprising an actuation element, wherein a second force measurement device is attached to the actuation element.
Example 9. The valve repair device according to any one of examples 1-8, wherein the valve repair device further comprises a coaptation element.
Example 10. The valve repair device according to any one of examples 1-9, wherein the valve repair device further comprises a cap, wherein the at least one paddle is attached to the cap such that movement of the cap causes the at least one paddle to move between the open position and the closed position.
Example 11. The valve repair device according to any one of examples 1-10, wherein the valve repair device further comprises:
   a shaft;
   a coupler that is attached to and movable along the shaft; and
   wherein the at least one paddle is attached to the coupler such that movement of the coupler along the shaft causes the at least one paddle to move between the open position and the closed position.
Example 12. A valve repair system, comprising:
   a delivery device comprising an actuation element and a force measurement device, wherein the force measurement device is attached to the actuation element;
   a valve repair device comprising:
   a cap;
at least one paddle attached to the cap, wherein the at least one paddle is movable between an open position and a closed position, wherein the at least one paddle is configured to attach the valve repair device to a native valve; and
   wherein the actuation element of the delivery device is configured to engage the cap to move the cap and cause the at least one paddle to move between the open position and the closed position.
Example 13. The valve repair system according to example 12, wherein the force measurement device is configured to measure a force applied to the actuation element when the valve repair device is attached to the native valve and the at least one paddle is in the closed position.
Example 14. The valve repair system according to any one of examples 12-13, wherein the force measurement device comprises a strain gauge and a wire attached to the strain gauge.
Example 15. The valve repair system according to any one of examples 12-14, wherein the valve repair device further comprises at least one gripping member configured to capture the native valve.
Example 16. The valve repair system according to example 15, wherein the valve repair device further comprises a second force measurement device that is attached to the at least one gripping member, wherein the second force measurement device is configured to measure a force provided to the at least one gripping member when the valve repair device is attached to the native valve.
Example 17. The valve repair system according to example 16, wherein the at least one gripping member comprises a clasp having:
   a fixed arm attached to the at least one paddle;
   a movable arm; and
   a hinge portion hingeably connecting the fixed arm to the movable arm.
Example 18. The valve repair system according to example 17, wherein the second force measurement device is attached to the movable arm of the clasp.
Example 19. The valve repair system according to example 17, wherein the second force measurement device is attached to the hinge portion of the clasp.
Example 20. The valve repair system according to example 16, wherein the second force measurement device comprises a strain gauge.
Example 21. The valve repair system according to example 20, wherein the second force measurement device comprises a wire attached to the strain gauge, and wherein the wire comprises a weakened portion that is configured to allow a user to break the wire after attaching the valve repair device to the native valve.
Example 22. The valve repair system according to example 16, wherein the at least one gripping member comprises a first gripping member and a second gripping member, wherein the second force measurement device is attached to the first gripping member and a third force measurement device is attached to the second gripping member.
Example 23. A valve repair system, comprising:
   a delivery device comprising a paddle control mechanism and a force measurement device, wherein the force measurement device is attached to the paddle control mechanism;
   a valve repair device comprising:
   a shaft;
a coupler that is attached to and movable along the shaft;
at least one paddle attached to the coupler, wherein the at least one paddle is movable between an open position and a closed position, wherein the at least one paddle is configured to attach the valve repair device to a native valve; and
   wherein the paddle control mechanism of the delivery device is configured to engage the coupler to move the coupler along the shaft and cause the at least one paddle to move between the open position and the closed position.
Example 24. The valve repair system according to example 23, wherein the force measurement device is configured to measure a force applied to the paddle control mechanism when the valve repair device is attached to the native valve and the at least one paddle is in the closed position.
Example 25. The valve repair system according to any one of examples 23-24, wherein the force measurement device comprises a strain gauge and a wire attached to the strain gauge.
Example 26. The valve repair system according to any one of examples 23-25, wherein the valve repair device further comprises at least one gripping member configured to capture the native valve.
Example 27. The valve repair system according to example 26, wherein the valve repair device further comprises a second force measurement device that is attached to the at least one gripping member, wherein the second force measurement device is configured to measure a force provided to the at least one gripping member when the valve repair device is attached to the native valve.
Example 28. The valve repair system according to example 27, wherein the at least one gripping member comprises a clasp having:
   a fixed arm attached to the at least one paddle;
   a movable arm; and
   a hinge portion hingeably connecting the fixed arm to the movable arm.
Example 29. The valve repair system according to example 28, wherein the second force measurement device is attached to the movable arm of the clasp.
Example 30. The valve repair system according to example 28, wherein the second force measurement device is attached to the hinge portion of the clasp.
Example 31. The valve repair system according to example 27, wherein the second force measurement device comprises a strain gauge.
Example 32. The valve repair system according to example 31, wherein the second force measurement device comprises a wire attached to the strain gauge, and wherein the wire comprises a weakened portion that is configured to allow a user to break the wire after attaching the valve repair device to the native valve.
Example 33. The valve repair system according to example 27, wherein the at least one gripping member comprises a first gripping member and a second gripping member, wherein the second force measurement device is attached to the first gripping member and a third force measurement device is attached to the second gripping member.
Example 34. A method for determining if a tension force provided to a native valve of a subject due to an attachment between a valve repair device and the native valve is at a desired level, the method comprising:
   delivering the valve repair device to the native valve of the subject with a delivery device, wherein at least one of the valve repair device and the delivery device comprise a force measurement device;
   attaching the valve repair device to the native valve of the subject;
   detecting a force applied to at least one of the valve repair device and the delivery device via the force measurement device; and
   determining if a tension force applied to the native valve is at the desired level based on the detected force by the force measurement device.
Example 35. The method according to example 34, further comprising:
detaching the valve repair device from the native valve if the tension force is not at the desired level, reattaching the valve repair device to the native valve;
redetecting the force applied to at least one of the valve repair device and the delivery device via the force measurement device; and
determining if the tension force applied to the native valve is at the desired level based on the redetected force by the force measurement device.
Example 36. The method according to any one of examples 34-35, wherein the valve repair device comprises:
at least one paddle movable between an open position and a closed position, wherein the at least one paddle is configured to attach the valve repair device to the native valve;
at least one gripping member configured to capture the native valve; and
the force measurement device attached to the at least one gripping member.
Example 37. The method according to example 36, wherein the at least one gripping member comprises a clasp having:
   a fixed arm attached to the at least one paddle;
   a movable arm; and
   a hinge portion hingeably connecting the fixed arm to the movable arm.
Example 38. The method according to example 37, wherein the force measurement device is attached to the movable arm of the clasp.
Example 39 The method according to example 37, wherein the force measurement device is attached to the hinge portion of the clasp.
Example 40. The method according to any one of examples 34-39, wherein the force measurement device comprises a strain gauge.
Example 41. The method according to example 40, wherein the detected force is detected via an ohmmeter.
Example 42. The method according to any one of examples 34-41, wherein the delivery device comprises an actuation element, and wherein the force measurement device is attached to the actuation element.
Example 43. The method according to example 42, wherein the valve repair device comprises:
a cap;
at least one paddle attached to the cap, wherein the at least one paddle is movable between an open position and a closed position, wherein the at least one paddle is configured to attach the valve repair device to the native valve; and
   wherein the actuation element of the delivery device is configured to engage the cap to move the cap and cause the at least one paddle to move between the open position and the closed position.
Example 44. The method according to any one of examples 34-43, wherein the delivery device comprising a paddle control mechanism, and wherein the force measurement device is attached to the paddle control mechanism.
Example 45. The method according to example 44, wherein the valve repair device comprises:
a shaft;
a coupler that is attached to and movable along the shaft;
at least one paddle attached to the coupler, wherein the at least one paddle is movable between an open position and a closed position, wherein the at least one paddle is configured to attach the valve repair device to the native valve; and
   wherein the paddle control mechanism of the delivery device is configured to engage the coupler to move the coupler along the shaft and cause the at least one paddle to move between the open position and the closed position.
Example 46. The valve repair device of any one of examples 34-45, wherein the force measurement device is integrally formed with one or more component of the valve repair device.
Example 47. The valve repair device of example 46, wherein the force measurement forms a portion of the at least one gripping member.
Example 48. The valve repair device of example 47, wherein the force measurement device forms at least a portion of a hinge portion of the at least one gripping member.
Example 49. The valve repair device of example 46, wherein the force measurement forms a portion of the at least one paddle.
Example 50. The valve repair device of example 49, wherein the force measurement device forms at least a portion of a hinge portion of the at least one paddle.

The techniques, methods, operations, steps, etc. described or suggested herein or in the references incorporated herein can be performed on a living subject (e.g., human, other animal, etc.) or on a simulation, such as a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Any of the various systems, assemblies, devices, components, apparatuses, etc. in this disclosure can be sterilized (e.g., with heat, radiation, ethylene oxide, hydrogen peroxide, etc.) to ensure they are safe for use with subjects, and the methods herein can comprise (or additional methods comprise or consist of) sterilization of the associated system, device, component, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A method for determining if a tension force provided to a native valve of a subject due to an attachment between a valve repair device and the native valve is at a desired level, the method comprising:
delivering the valve repair device to the native valve with a delivery device, wherein at least one of the valve repair device and the delivery device comprise a force measurement device;
attaching the valve repair device to the native valve of the subject;
detecting the tension force applied to at least one of the valve repair device and the delivery device via the force measurement device; and
determining if the tension force applied to the native valve is at the desired level based on the detected force by the force measurement device.

2. The method according to claim 1, further comprising:
detaching the valve repair device from the native valve if the tension force is not at the desired level,
reattaching the valve repair device to the native valve;
redetecting the force applied to at least one of the valve repair device and the delivery device via the force measurement device; and
determining if the tension force applied to the native valve is at the desired level based on the redetected force by the force measurement device.

3. The method according to claim 1, wherein the valve repair device comprises:
at least one paddle movable between an open position and a closed position, wherein the at least one paddle is configured to attach the valve repair device to the native valve;
at least one gripping member configured to capture the native valve; and
the force measurement device attached to the at least one gripping member.

4. The method according to claim 3, wherein the force measurement device is attached to one or more of a movable arm of the gripping member and hinge portion of the gripping member.

5. The method according to claim 1, wherein the force measurement device comprises a strain gauge.

6. The method according to claim 5, wherein the detected force is detected via an ohmmeter.

7. The method according to claim 1, wherein the delivery device comprises an actuation element, and wherein the force measurement device is attached to the actuation element.

8. The method according to claim 1, wherein the delivery device comprising a paddle control mechanism, and wherein the force measurement device is attached to the paddle control mechanism.

9. A valve repair device for repairing a native valve of a heart, the valve repair device comprising:
at least one paddle movable between an open position and a closed position, wherein the at least one paddle is configured to attach the valve repair device to the native valve;
at least one gripping member configured to capture the native valve;
a force measurement device attached to the at least one gripping member; and
wherein the force measurement device is configured to detect a force provided to the at least one gripping member when the valve repair device is attached to the native valve.

10. The valve repair device according to claim 9, wherein the force measurement device is attached to one or more of a movable arm of the gripping member and a hinge portion of the gripping member.

11. The valve repair device according to claim 9, wherein the force measurement device comprises a wire attached to a strain gauge, and wherein the wire comprises a weakened portion that is configured to allow a user to break the wire after attaching the valve repair device to the native valve.

12. The valve repair device according to claim 9, further comprising an actuation element, wherein a second force measurement device is attached to the actuation element.

13. A valve repair system, comprising:
a delivery device comprising an actuation element and a force measurement device, wherein the force measurement device is attached to the actuation element;
a valve repair device comprising:
a cap;
at least one paddle attached to the cap, wherein the at least one paddle is movable between an open position and a closed position, wherein the at least one paddle is configured to attach the valve repair device to a native valve; and
wherein the actuation element of the delivery device is configured to engage the cap to move the cap and cause the at least one paddle to move between the open position and the closed position.

14. The valve repair system according to claim 13, wherein the force measurement device is configured to measure a force applied to the actuation element when the valve repair device is attached to the native valve and the at least one paddle is in the closed position.

15. The valve repair system according to claim 14, wherein the valve repair device further comprises at least one gripping member configured to capture the native valve and wherein the valve repair device further comprises a second force measurement device that is attached to the at least one gripping member, wherein the second force measurement device is configured to measure the force provided to the at least one gripping member when the valve repair device is attached to the native valve.

16. A valve repair system, comprising:
a delivery device comprising a paddle control mechanism and a force measurement device, wherein the force measurement device is attached to the paddle control mechanism;
a valve repair device comprising:
a shaft;
a coupler that is attached to and movable along the shaft;
at least one paddle attached to the coupler, wherein the at least one paddle is movable between an open position and a closed position, wherein the at least one paddle is configured to attach the valve repair device to a native valve; and
wherein the paddle control mechanism of the delivery device is configured to engage the coupler to move the coupler along the shaft and cause the at least one paddle to move between the open position and the closed position.

17. The valve repair system according to claim 16, wherein the force measurement device is configured to measure a force applied to the paddle control mechanism when the valve repair device is attached to the native valve and the at least one paddle is in the closed position.

18. The valve repair system according to claim 13, wherein the valve repair device further comprises a second force measurement device that is attached to at least one gripping member, wherein the second force measurement device is configured to measure the force provided to the at least one gripping member when the valve repair device is attached to the native valve.

19. The valve repair system according to claim 18, wherein the second force measurement device is attached to at least one of a movable arm of the gripping member and a hinge portion of the gripping member.

20. The valve repair system according to claim 19, wherein the second force measurement device comprises a strain gauge.
